SEQUENCE LISTING (78 sequences)

SEQ ID NO: 1 - protein, 673 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEIL

SEQ ID NO: 2 - protein, 103 aa, Homo sapiens
EQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYD

SEQ ID NO: 3 - protein, 102 aa, Homo sapiens
NGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVL

SEQ ID NO: 4 - protein, 126 aa, Homo sapiens
PHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLS

SEQ ID NO: 5 - protein, 108 aa, Homo sapiens
PYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYS

SEQ ID NO: 6 - protein, 108 aa, Homo sapiens
SLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEK

SEQ ID NO: 7 - protein, 110 aa, Homo sapiens
CGNQLQVHLSPDADAYSPGQTVSLNMATGMDSWVALAAVDLHMKTLLPVSKPEIRSYFPESWLWEVHLVPRRKQLQFALPDSLTTWEIQGVGISNTGICVADTVKAKVFK

SEQ ID NO: 8 - protein, 206 aa, Homo sapiens
EQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLP

SEQ ID NO: 9 - protein, 448 aa, Homo sapiens
HFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEIL

SEQ ID NO: 10 - protein, 999 aa, Homo sapiens
TLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 11 - protein, 74 aa, Homo sapiens
TLQKKIEEIAAKYKHSVVKKCCYDGACVNNDETCEQRAARISLGPRCIKAFTECCVVASQLRANISHKDMQLGR

SEQ ID NO: 12 - protein, 147 aa, Homo sapiens
ADCGQMQEELDLTISAETRKQTACKPEIAYAYKVSITSITVENVFVKYKATLLDIYKTGEAVAEKDSEITFIKKVTCTNAELVKGRQYLIMGKEALQIKYNASFRYIYPLDSLTWIEYWPRDTTCSSCQAFLANLDEFAEDIFLNGC

SEQ ID NO: 13 - protein, 1676 aa, Homo sapiens
MGLLGILCFLIFLGKTWGQEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 14 - protein, 1676 aa, Macaca fascicularis
MGLLGILCFLIFLGKTWGQEQTYVISAPKIFRVGASENIVIQVYGYTEAFDATISIKSYPDKKFSYSSGHVHLSSENKFQNSAVLTIQPKQLPGGQNQVSYVYLEVVSKHFSKSKKIPITYDNGFLFIHTDKPVYTPDQSVKVRVYSLNDDLKPAKRETVLTFIDPEGSEIDMVEEIDHIGIISFPDFKIPSNPRYGMWTIQAKYKEDFSTTGTAFFEVKEYVLPHFSVSVEPESNFIGYKNFKNFEITIKARYFYNKVVTEADVYITFGIREDLKDDQKEMMQTAMQNTMLINGIAEVTFDSETAVKELSYYSLEDLNNKYLYIAVTVIESTGGFSEEAEIPGIKYVLSPYKLNLVATPLFLKPGIPYSIKVQVKDALDQLVGGVPVTLNAQTIDVNQETSDLEPRKSVTRVDDGVASFVVNLPSGVTVLEFNVKTDAPDLPDENQAREGYRAIAYSSLSQSYLYIDWTDNHKALLVGEYLNIIVTPKSPYIDKITHYNYLILSKGKIIHFGTREKLSDASYQSINIPVTQNMVPSSRLLVYYIVTGEQTAELVSDSVWLNIEEKCGNQLQVHLSPDADTYSPGQTVSLNMVTGMDSWVALTAVDSAVYGVQRRAKKPLERVFQFLEKSDLGCGAGGGLNNANVFHLAGLTFLTNANADDSQENDEPCKEIIRPRRMLQEKIEEIAAKYKHLVVKKCCYDGVRINHDETCEQRAARISVGPRCVKAFTECCVVASQLRANNSHKDLQLGRLHMKTLLPVSKPEIRSYFPESWLWEVHLVPRRKQLQFALPDSVTTWEIQGVGISNSGICVADTIKAKVFKDVFLEMNIPYSVVRGEQVQLKGTVYNYRTSGMQFCVKMSAVEGICTSESPVIDHQGTKSSKCVRQKVEGSSNHLVTFTVLPLEIGLQNINFSLETSFGKEILVKSLRVVPEGVKRESYSGITLDPRGIYGTISRRKEFPYRIPLDLVPKTEIKRILSVKGLLVGEILSAVLSREGINILTHLPKGSAEAELMSVVPVFYVFHYLETGNHWNIFHSDPLIEKRNLEKKLKEGMVSIMSYRNADYSYSVWKGGSASTWLTAFALRVLGQVHKYVEQNQNSICNSLLWLVENYQLDNGSFKENSQYQPIKLQGTLPVEARENSLYLTAFTVIGIRKAFDICPLVKINTALIKADTFLLENTLPAQSTFTLAISAYALSLGDKTHPQFRSIVSALKREALVKGNPPIYRFWKDSLQHKDSSVPNTGTARMVETTAYALLTSLNLKDINYVNPIIKWLSEEQRYGGGFYSTQDTINAIEGLTEYSLLVKQLRLNMDIDVAYKHKGPLHNYKMTDKNFLGRPVEVLLNDDLVVSTGFGSGLATVHVTTVVHKTSTSEEVCSFYLKIDTQDIEASHYRGYGNSDYKRIVACASYKPSKEESSSGSSHAVMDISLPTGINANEEDLKALVEGVDQLFTDYQIKDGHVILQLNSIPSSDFLCVRFRIFELFEVGFLSPATFTVYEYHRPDKQCTMFYSTSNIKIQKVCEGATCKCIEADCGQMQKELDLTISAETRKQTACNPEIAYAYKVIITSITTENVFVKYKATLLDIYKTGEAVAEKDSEITFIKKVTCTNAELVKGRQYLIMGKEALQIKYNFTFRYIYPLDSLTWIEYWPRDTTCSSCQAFLANLDEFAEDIFLNGC

SEQ ID NO: 15 - protein, 126 aa, synthetic construct
  REGION 1..126: note = An artificially synthesized sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGVHIKYMIQYYYGAMDVWGQGTTVTVSS

SEQ ID NO: 16 - protein, 107 aa, synthetic construct
  REGION 1..107: note = An artificially synthesized sequence
DIQMTQSPSSLSASVGDRVTITCRASQSIEDDLAWYQQKPGKAPKLLIYEASSLQSGVPSRFSGSGSGTDFTLTISSLQPEDFATYYCQQHDGSPLTFGQGTKVEIK

SEQ ID NO: 17 - protein, 127 aa, synthetic construct
  REGION 1..127: note = An artificially synthesized sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDMSEFLGWSNYYSYPMDVWGQGTTVTVSS

SEQ ID NO: 18 - protein, 107 aa, synthetic construct
  REGION 1..107: note = An artificially synthesized sequence
DIQMTQSPSSLSASVGDRVTITCRASQSIEDDLAWYQQKPGKAPKLLIYHASSLQSGVPSRFSGSGSGTDFTLTISSLQPEDFATYYCQQSDNSPYTFGQGTKVEIK

SEQ ID NO: 19 - protein, 123 aa, synthetic construct
  REGION 1..123: note = An artificially synthesized sequence
QVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGDQIWYDQWYYFDMWGQGTLVTVSS

SEQ ID NO: 20 - protein, 107 aa, synthetic construct
  REGION 1..107: note = An artificially synthesized sequence
DIQMTQSPSSLSASVGDRVTITCRASQSIEDDLAWYQQKPGKAPKLLIYEASNLQSGVPSRFSGSGSGTDFTLTISSLQPEDFATYYCQQYDSSPLTFGQGTKVEIK

SEQ ID NO: 21 - protein, 122 aa, synthetic construct
  REGION 1..122: note = An artificially synthesized sequence
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARDPYYSYPWSTFDYWGQGTLVTVSS

SEQ ID NO: 22 - protein, 107 aa, synthetic construct
  REGION 1..107: note = An artificially synthesized sequence
DIQMTQSPSSLSASVGDRVTITCRASQSIEDDLAWYQQKPGKAPKLLIYEASTLQSGVPSRFSGSGSGTDFTLTISSLQPEDFATYYCQQSNNLPLTFGQGTKVEIK

SEQ ID NO: 23 - protein, 119 aa, synthetic construct
  REGION 1..119: note = An artificially synthesized sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGGWWGGALDYWGQGTLVTVSS

SEQ ID NO: 24 - protein, 107 aa, synthetic construct
  REGION 1..107: note = An artificially synthesized sequence
DIQMTQSPSSLSASVGDRVTITCRASQSIEDDLAWYQQKPGKAPKLLIYHASSLQSGVPSRFSGSGSGTDFTLTISSLQPEDFATYYCQQSDSYPLTFGQGTKVEIK

SEQ ID NO: 25 - protein, 119 aa, synthetic construct
  REGION 1..119: note = An artificially synthesized sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARQLYGYYELDIWGQGTLVTVSS

SEQ ID NO: 26 - protein, 107 aa, synthetic construct
  REGION 1..107: note = An artificially synthesized sequence
DIQMTQSPSSLSASVGDRVTITCRASQSISDDLAWYQQKPGKAPKLLIYEASSLQSGVPSRFSGSGSGTDFTLTISSLQPEDFATYYCQQYNSYPLTFGQGTKVEIK

SEQ ID NO: 27 - protein, 122 aa, synthetic construct
  REGION 1..122: note = An artificially synthesized sequence
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARYYVWLGGPTYMDYWGQGTLVTVSS

SEQ ID NO: 28 - protein, 107 aa, synthetic construct
  REGION 1..107: note = An artificially synthesized sequence
DIQMTQSPSSLSASVGDRVTITCRASQSIEDDLAWYQQKPGKAPKLLIYHASTLQSGVPSRFSGSGSGTDFTLTISSLQPEDFATYYCQQSDGYPLTFGQGTKVEIK

SEQ ID NO: 29 - protein, 122 aa, synthetic construct
  REGION 1..122: note = An artificially synthesized sequence
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARHDPTWYSTGYFDYWGQGTLVTVSS

SEQ ID NO: 30 - protein, 107 aa, synthetic construct
  REGION 1..107: note = An artificially synthesized sequence
DIQMTQSPSSLSASVGDRVTITCRASQSISDDLAWYQQKPGKAPKLLIYEASNLQSGVPSRFSGSGSGTDFTLTISSLQPEDFATYYCQQYDSYPYTFGQGTKVEIK

SEQ ID NO: 31 - protein, 122 aa, synthetic construct
  REGION 1..122: note = An artificially synthesized sequence
QVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCARTGMMYWGIFDVWGQGTLVTVSS

SEQ ID NO: 32 - protein, 107 aa, synthetic construct
  REGION 1..107: note = An artificially synthesized sequence
DIQMTQSPSSLSASVGDRVTITCRASQSIEDDLAWYQQKPGKAPKLLIYHASTLQSGVPSRFSGSGSGTDFTLTISSLQPEDFATYYCQQHDSYPLTFGQGTKVEIK

SEQ ID NO: 33 - protein, 325 aa, synthetic construct
  REGION 1..325: note = An artificially synthesized sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRGGPKVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSRLTVDKSRWQEGNVFSCSVMHEALHNHYTQKSLSLSL

SEQ ID NO: 34 - protein, 325 aa, synthetic construct
  REGION 1..325: note = An artificially synthesized sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFRGGPKVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQESLSLSL

SEQ ID NO: 35 - protein, 107 aa, synthetic construct
  REGION 1..107: note = An artificially synthesized sequence
DIQMTQSPSSLSASVGDRVTITCRASQSIHDDLAWYQQKPGKAPKLLIYEASNLQSGVPSRFSGSGSGTDFTLTISSLQPEDFATYYCQQYSSYPHTFGQGTKVEIK

SEQ ID NO: 36 - protein, 11 aa, synthetic construct
  REGION 1..11: note = An artificially synthesized sequence
RASQSIEDDLA

SEQ ID NO: 37 - protein, 11 aa, synthetic construct
  REGION 1..11: note = An artificially synthesized sequence
RASQSISDDLA

SEQ ID NO: 38 - protein, 7 aa, synthetic construct
  REGION 1..7: note = An artificially synthesized sequence
HASSLQS

SEQ ID NO: 39 - protein, 7 aa, synthetic construct
  REGION 1..7: note = An artificially synthesized sequence
EASNLQS

SEQ ID NO: 40 - protein, 7 aa, synthetic construct
  REGION 1..7: note = An artificially synthesized sequence
EASSLQS

SEQ ID NO: 41 - protein, 7 aa, synthetic construct
  REGION 1..7: note = An artificially synthesized sequence
HASTLQS

SEQ ID NO: 42 - protein, 9 aa, synthetic construct
  REGION 1..9: note = An artificially synthesized sequence
QQSDNSPYT

SEQ ID NO: 43 - protein, 9 aa, synthetic construct
  REGION 1..9: note = An artificially synthesized sequence
QQYDSSPLT

SEQ ID NO: 44 - protein, 9 aa, synthetic construct
  REGION 1..9: note = An artificially synthesized sequence
QQSDSYPLT

SEQ ID NO: 45 - protein, 9 aa, synthetic construct
  REGION 1..9: note = An artificially synthesized sequence
QQYNSYPLT

SEQ ID NO: 46 - protein, 9 aa, synthetic construct
  REGION 1..9: note = An artificially synthesized sequence
QQSDGYPLT

SEQ ID NO: 47 - protein, 9 aa, synthetic construct
  REGION 1..9: note = An artificially synthesized sequence
QQYDSYPYT

SEQ ID NO: 48 - protein, 9 aa, synthetic construct
  REGION 1..9: note = An artificially synthesized sequence
QQHDSYPLT

SEQ ID NO: 49 - protein, 328 aa, synthetic construct
  REGION 1..328: note = An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPYLDSDGSFFLYSKLTVDKSRWQQGNVFSCSVMHEALHYHYTQKSLSLSP

SEQ ID NO: 50 - protein, 328 aa, synthetic construct
  REGION 1..328: note = An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRGGPKVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPYLDSDGSFFLYSKLTVDKSRWQQGNVFSCSVMHEALHYHYTQESLSLSP

SEQ ID NO: 51 - protein, 328 aa, synthetic construct
  REGION 1..328: note = An artificially synthesized sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDVLMISRTPEVTCVVVDVSFEDPEVKFNWYVDGVEVHNAKTKPREEPYNSTYRVVSVLPVLHQDWLNGKEYKCKVSNKALPAPIEKTISKAKGQPREPQVYTLPPSRKELTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 52 - protein, 122 aa, synthetic construct
  REGION 1..122: note = An artificially synthesized sequence
QVQLQESGPGLVKPSETLSLTCAVSGYSISSGYYWGWIRQPPGKGLEWIGSIYHSGSTYYNKKLKSRVTISVDTSKNQFSLKLSSVTAADTAVYYCARHDPTWYHHGYFDYWGQGTLVTVSS

SEQ ID NO: 53 - protein, 107 aa, synthetic construct
  REGION 1..107: note = An artificially synthesized sequence
DIQMTQSPSSLSASVGDRVTITCRASQSIHNDLAWYQQKPGKAPKLLIYEASELQSGVPSRFSGSGSGTDFTLTISSLQPEDFATYYCQHYSSYPYTFGQGTKVEIK

SEQ ID NO: 54 - protein, 119 aa, synthetic construct
  REGION 1..119: note = An artificially synthesized sequence
QVQLVQSGAEVKKPGSSVKVSCKASGGTFSSYSISWVRQAPGQGLEWMGGIIPIFGTANYAQKFQGRVTITADESTSTAYMELSSLRSEDTAVYYCARQLYGYYELDIWGQGTLVTVSS

SEQ ID NO: 55 - protein, 450 aa, synthetic construct
  REGION 1..450: note = An artificially synthesized sequence
QVQLQESGPGLVKPSETLSLTCAVSGYSISSGYYWGWIRQPPGKGLEWIGSIYHSGSTYYNKKLKSRVTISVDTSKNQFSLKLSSVTAADTAVYYCARHDPTWYHHGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 56 - protein, 447 aa, synthetic construct
  REGION 1..447: note = An artificially synthesized sequence
QVQLVQSGAEVKKPGSSVKVSCKASGGTFSSYSISWVRQAPGQGLEWMGGIIPIFGTANYAQKFQGRVTITADESTSTAYMELSSLRSEDTAVYYCARQLYGYYELDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQESLSLSP

SEQ ID NO: 57 - protein, 214 aa, synthetic construct
  REGION 1..214: note = An artificially synthesized sequence
DIQMTQSPSSLSASVGDRVTITCRASQSIHNDLAWYQQKPGKAPKLLIYEASELQSGVPSRFSGSGSGTDFTLTISSLQPEDFATYYCQHYSSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 450 aa, synthetic construct
  REGION 1..450: note = An artificially synthesized sequence
QVQLQESGPGLVKPSETLSLTCAVSGYSISSGYYWGWIRQPPGKGLEWIGSIYHSGSTYYNKKLKSRVTISVDTSKNQFSLKLSSVTAADTAVYYCARHDPTWYHHGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDVLMISRTPEVTCVVVDVSFEDPEVKFNWYVDGVEVHNAKTKPREEPYNSTYRVVSVLPVLHQDWLNGKEYKCKVSNKALPAPIEKTISKAKGQPREPQVYTLPPSRKELTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 59 - protein, 447 aa, synthetic construct
  REGION 1..447: note = An artificially synthesized sequence
QVQLVQSGAEVKKPGSSVKVSCKASGGTFSSYSISWVRQAPGQGLEWMGGIIPIFGTANYAQKFQGRVTITADESTSTAYMELSSLRSEDTAVYYCARQLYGYYELDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDVLMISRTPEVTCVVVDVSFEDPEVKFNWYVDGVEVHNAKTKPREEPYNSTYRVVSVLPVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTREELSLSP

SEQ ID NO: 60 - protein, 450 aa, synthetic construct
  REGION 1..450: note = An artificially synthesized sequence
QVQLQESGPGLVKPSETLSLTCAVSGYSISSGYYWGWIRQPPGKGLEWIGSIYHSGSTYYNKKLKSRVTISVDTSKNQFSLKLSSVTAADTAVYYCARHDPTWYHHGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDVLMISRTPEVTCVVVDVSFEDPEVKFNWYVDGVEVHNAKTKPREEQYNSTYRVVSVLPVLHQDWLNGKEYKCKVSNKAAPKPIEKTISKAKGQPREPQVYTLPPSRKELTKNQVSLTCLVKGFYPSDIAVEWESNGQPENNYKTTPPVLDSDGSFFLYSKLTVDKSRWQQGNVFSCSVMHEALHAHYTRKELSLSP

SEQ ID NO: 61 - protein, 447 aa, synthetic construct
  REGION 1..447: note = An artificially synthesized sequence
QVQLVQSGAEVKKPGSSVKVSCKASGGTFSSYSISWVRQAPGQGLEWMGGIIPIFGTANYAQKFQGRVTITADESTSTAYMELSSLRSEDTAVYYCARQLYGYYELDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLAGPSVFLFPPKPKDVLMISRTPEVTCVVVDVSFEDPEVKFNWYVDGVEVHNAKTKPREEQYNSTYRVVSVLPVLHQDWLNGKEYKCKVSNKAAPKPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHAHYTREELSLSP

SEQ ID NO: 62 - protein, 1680 aa, Mus musculus
MGLWGILCLLIFLDKTWGQEQTYVISAPKILRVGSSENVVIQVHGYTEAFDATLSLKSYPDKKVTFSSGYVNLSPENKFQNAALLTLQPNQVPREESPVSHVYLEVVSKHFSKSKKIPITYNNGILFIHTDKPVYTPDQSVKIRVYSLGDDLKPAKRETVLTFIDPEGSEVDIVEENDYTGIISFPDFKIPSNPKYGVWTIKANYKKDFTTTGTAYFEIKEYVLPRFSVSIELERTFIGYKNFKNFEITVKARYFYNKVVPDAEVYAFFGLREDIKDEEKQMMHKATQAAKLVDGVAQISFDSETAVKELSYNSLEDLNNKYLYIAVTVTESSGGFSEEAEIPGVKYVLSPYTLNLVATPLFVKPGIPFSIKAQVKDSLEQAVGGVPVTLMAQTVDVNQETSDLETKRSITHDTDGVAVFVLNLPSNVTVLKFEIRTDDPELPEENQASKEYEAVAYSSLSQSYIYIAWTENYKPMLVGEYLNIMVTPKSPYIDKITHYNYLILSKGKIVQYGTREKLFSSTYQNINIPVTQNMVPSARLLVYYIVTGEQTAELVADAVWINIEEKCGNQLQVHLSPDEYVYSPGQTVSLDMVTEADSWVALSAVDRAVYKVQGNAKRAMQRVFQALDEKSDLGCGAGGGHDNADVFHLAGLTFLTNANADDSHYRDDSCKEILRSKRNLHLLRQKIEEQAAKYKHSVPKKCCYDGARVNFYETCEERVARVTIGPLCIRAFNECCTIANKIRKESPHKPVQLGRIHIKTLLPVMKADIRSYFPESWLWEIHRVPKRKQLQVTLPDSLTTWEIQGIGISDNGICVADTLKAKVFKEVFLEMNIPYSVVRGEQIQLKGTVYNYMTSGTKFCVKMSAVEGICTSGSSAASLHTSRPSRCVFQRIEGSSSHLVTFTLLPLEIGLHSINFSLETSFGKDILVKTLRVVPEGVKRESYAGVILDPKGIRGIVNRRKEFPYRIPLDLVPKTKVERILSVKGLLVGEFLSTVLSKEGINILTHLPKGSAEAELMSIAPVFYVFHYLEAGNHWNIFYPDTLSKRQSLEKKIKQGVVSVMSYRNADYSYSMWKGASASTWLTAFALRVLGQVAKYVKQDENSICNSLLWLVEKCQLENGSFKENSQYLPIKLQGTLPAEAQEKTLYLTAFSVIGIRKAVDICPTMKIHTALDKADSFLLENTLPSKSTFTLAIVAYALSLGDRTHPRFRLIVSALRKEAFVKGDPPIYRYWRDTLKRPDSSVPSSGTAGMVETTAYALLASLKLKDMNYANPIIKWLSEEQRYGGGFYSTQDTINAIEGLTEYSLLLKQIHLDMDINVAYKHEGDFHKYKVTEKHFLGRPVEVSLNDDLVVSTGYSSGLATVYVKTVVHKISVSEEFCSFYLKIDTQDIEASSHFRLSDSGFKRIIACASYKPSKEESTSGSSHAVMDISLPTGIGANEEDLRALVEGVDQLLTDYQIKDGHVILQLNSIPSRDFLCVRFRIFELFQVGFLNPATFTVYEYHRPDKQCTMIYSISDTRLQKVCEGAACTCVEADCAQLQAEVDLAISADSRKEKACKPETAYAYKVRITSATEENVFVKYTATLLVTYKTGEAADENSEVTFIKKMSCTNANLVKGKQYLIMGKEVLQIKHNFSFKYIYPLDSSTWIEYWPTDTTCPSCQAFVENLNNFAEDLFLNSCE

SEQ ID NO: 63 - protein, 5 aa, synthetic construct
  REGION 1..5: note = An artificially synthesized sequence
SYAIS

SEQ ID NO: 64 - protein, 5 aa, synthetic construct
  REGION 1..5: note = An artificially synthesized sequence
SYAMS

SEQ ID NO: 65 - protein, 6 aa, synthetic construct
  REGION 1..6: note = An artificially synthesized sequence
SGYYWG

SEQ ID NO: 66 - protein, 5 aa, synthetic construct
  REGION 1..5: note = An artificially synthesized sequence
DHYMD

SEQ ID NO: 67 - protein, 17 aa, synthetic construct
  REGION 1..17: note = An artificially synthesized sequence
GIIPIFGTANYAQKFQG

SEQ ID NO: 68 - protein, 17 aa, synthetic construct
  REGION 1..17: note = An artificially synthesized sequence
AISGSGGSTYYADSVKG

SEQ ID NO: 69 - protein, 16 aa, synthetic construct
  REGION 1..16: note = An artificially synthesized sequence
SIYHSGSTYYNPSLKS

SEQ ID NO: 70 - protein, 16 aa, synthetic construct
  REGION 1..16: note = An artificially synthesized sequence
SIYHSGSTYYNPSLKS

SEQ ID NO: 71 - protein, 19 aa, synthetic construct
  REGION 1..19: note = An artificially synthesized sequence
RTRNKANSYTTEYAASVKG

SEQ ID NO: 72 - protein, 18 aa, synthetic construct
  REGION 1..18: note = An artificially synthesized sequence
DMSEFLGWSNYYSYPMDV

SEQ ID NO: 73 - protein, 14 aa, synthetic construct
  REGION 1..14: note = An artificially synthesized sequence
GDQIWYDQWYYFDM

SEQ ID NO: 74 - protein, 10 aa, synthetic construct
  REGION 1..10: note = An artificially synthesized sequence
GGWWGGALDY

SEQ ID NO: 75 - protein, 10 aa, synthetic construct
  REGION 1..10: note = An artificially synthesized sequence
QLYGYYELDI

SEQ ID NO: 76 - protein, 13 aa, synthetic construct
  REGION 1..13: note = An artificially synthesized sequence
YYVWLGGPTYMDY

SEQ ID NO: 77 - protein, 13 aa, synthetic construct
  REGION 1..13: note = An artificially synthesized sequence
HDPTWYSTGYFDY

SEQ ID NO: 78 - protein, 11 aa, synthetic construct
  REGION 1..11: note = An artificially synthesized sequence
TGMMYWGIFDV

(19) United States
(12) Patent Application Publication
(10) Pub. No.: US 2023/0203144 A1
(43) Pub. Date: Jun. 29, 2023

(54) COMBINATION OF TWO OR MORE ANTI-C5 ANTIBODIES AND METHODS OF USE

(71) Applicant: Chugai Seiyaku Kabushiki Kaisha, Tokyo (JP)

(72) Inventors: Eriko MURATA, Kanagawa (JP); Shinya ISHII, Shizuoka (JP); Tomoyuki IGAWA, Shizuoka (JP); Yuji HORI, Shizuoka (JP); Norihito SHIBAHARA, Shizuoka (JP)

(21) Appl. No.: 18/059,677

(22) Filed: Nov. 29, 2022

Related U.S. Application Data
(62) Division of application No. 15/544,930, filed on Jul. 20, 2017, now abandoned.

(30) Foreign Application Priority Data
Jan. 22, 2015 (JP) 2015-010410

(51) Int. Cl. C07K 16/18; C07K 16/42; A61K 39/395

(52) U.S. Cl. CPC C07K 16/18; A61K 39/3955; C07K 16/42; A61K 2039/507

(57) ABSTRACT

The present invention provides a combination of two or more isolated or purified anti-C5 antibodies, wherein the isolated or purified anti-C5 antibodies bind to an epitope within the beta chain or alpha chain of C5 and wherein the isolated or purified anti-C5 antibodies to be combined do not compete with each other for binding to the epitope. Methods of using the combination for treating an individual having a complement-mediated disease or condition which involves excessive or uncontrolled activation of C5, or for enhancing the clearance of C5 from plasma in an individual, are also provided.

### TECHNICAL FIELD

The present invention relates to a combination of two or more anti-C5 antibodies and methods of using the same.

### BACKGROUND ART

The complement system plays a central role in the clearance of immune complexes and in immune responses to infectious agents, foreign antigens, virus-infected cells and tumor cells. There are about 25-30 complement proteins, which are found as a complex collection of plasma proteins and membrane cofactors. Complement components achieve their immune defensive functions by interacting in a series of intricate enzymatic cleavages and membrane binding events. The resulting complement cascades lead to the production of products with opsonic, immunoregulatory, and lytic functions.

Currently, it is widely accepted that the complement system can be activated through three distinct pathways: the classical pathway, the lectin pathway, and the alternative pathway. These pathways share many components, and while they differ in their initial steps, they converge and share the same terminal complement components (C5 through C9) responsible for the activation and destruction of target cells.

The classical pathway is normally activated by the formation of antigen-antibody complexes. Independently, the first step in activation of the lectin pathway is the binding of specific lectins such as mannan-binding lectin (MBL), H-ficolin, M-ficolin, L-ficolin and C-type lectin CL-11. In contrast, the alternative pathway spontaneously undergoes a low level of turnover activation, which can be readily amplified on foreign or other abnormal surfaces (bacteria, yeast, virally infected cells, or damaged tissue). These pathways converge at a point where complement component C3 is cleaved by an active protease to yield C3a and C3b.

C3a is an anaphylatoxin. C3b binds to bacterial and other cells, as well as to certain viruses and immune complexes, and tags them for removal from the circulation (the role known as opsonin). C3b also forms a complex with other components to form C5 convertase, which cleaves C5 into C5a and C5b.

C5 is a 190 kDa protein found in normal serum at approximately 80 micro g/ml (0.4 micro M). C5 is glycosylated with about 1.5-3% of its mass attributed to carbohydrate. Mature C5 is a heterodimer of 115 kDa alpha chain that is disulfide linked to 75 kDa beta chain. C5 is synthesized as a single chain precursor protein (pro-C5 precursor) of 1676 amino acids (See, e.g., PTL1 and PTL2). The pro-C5 precursor is cleaved to yield the beta chain as an amino terminal fragment and the alpha chain as a carboxyl terminal fragment. The alpha chain and the beta chain polypeptide fragments are connected to each other via disulfide bond and constitute the mature C5 protein.

Mature C5 is cleaved into the C5a and C5b fragments during activation of the complement pathways. C5a is cleaved from the alpha chain of C5 by C5 convertase as an amino terminal fragment comprising the first 74 amino acids of the alpha chain. The remaining portion of mature C5 is fragment C5b, which contains the rest of the alpha chain disulfide bonded to the beta chain. Approximately 20 % of the 11 kDa mass of C5a is attributed to carbohydrate.

C5a is another anaphylatoxin. C5b combines with C6, C7, C8 and C9 to form the membrane attack complex (MAC, C5b-9, terminal complement complex (TCC)) at the surface of the target cell. When sufficient numbers of MACs are inserted into target cell membranes, MAC pores are formed to mediate rapid osmotic lysis of the target cells.

As mentioned above, C3a and C5a are anaphylatoxins. They can trigger mast cell degranulation, which releases histamine and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract granulocytes such as neutrophils, eosinophils, basophils and monocytes to the site of complement activation.

The activity of C5a is regulated by the plasma enzyme carboxypeptidase N that removes the carboxy-terminal arginine from C5a forming C5a-des-Arg derivative. C5a-des-Arg exhibits only 1% of the anaphylactic activity and polymorphonuclear chemotactic activity as unmodified C5a.

While a properly functioning complement system provides a robust defense against infecting microbes, inappropriate regulation or activation of complement has been implicated in the pathogenesis of a variety of disorders including, e.g., rheumatoid arthritis (RA); lupus nephritis; ischemia-reperfusion injury; paroxysmal nocturnal hemoglobinuria (PNH); atypical hemolytic uremic syndrome (aHUS); dense deposit disease (DDD); macular degeneration (e.g., age-related macular degeneration (AMD)); hemolysis, elevated liver enzymes, and low platelets (HELLP) syndrome; thrombotic thrombocytopenic purpura (TTP); spontaneous fetal loss; Pauci-immune vasculitis; epidermolysis bullosa; recurrent fetal loss; multiple sclerosis (MS); traumatic brain injury; and injury resulting from myocardial infarction, cardiopulmonary bypass and hemodialysis (See, e.g., NPL1). Therefore, inhibition of excessive or uncontrolled activations of the complement cascade can provide clinical benefits to patients with such disorders.

Paroxysmal nocturnal hemoglobinuria (PNH) is an uncommon blood disorder, wherein red blood cells are compromised and are thus destroyed more rapidly than normal red blood cells. PNH results from the clonal expansion of hematopoietic stem cells with somatic mutations in the PIG-A (phosphatidylinositol glycan class A) gene which is located on the X chromosome. Mutations in PIG-A lead to an early block in the synthesis of glycosylphosphatidylinositol (GPI), a molecule which is required for the anchor of many proteins to cell surfaces. Consequently, PNH blood cells are deficient in GPI-anchored proteins, which include complement-regulatory proteins CD55 and CD59. Under normal circumstances, these complement-regulatory proteins block the formation of MAC on cell surfaces, thereby preventing erythrocyte lysis. The absence of those proteins causes complement-mediated hemolysis in PNH.

PNH is characterized by hemolytic anemia (a decreased number of red blood cells), hemoglobinuria (the presence of hemoglobin in urine, particularly evident after sleeping), and hemoglobinemia (the presence of hemoglobin in the bloodstream). PNH-afflicted individuals are known to have paroxysms, which are defined here as incidences of dark-colored urine. Hemolytic anemia is due to intravascular destruction of red blood cells by complement components. Other known symptoms include dysphasia, fatigue, erectile dysfunction, thrombosis and recurrent abdominal pain.

Eculizumab is a humanized monoclonal antibody directed against the complement protein C5, and the first therapy approved for the treatment of paroxysmal nocturnal hemoglobinuria (PNH) and atypical hemolytic uremic syndrome (aHUS) (See, e.g., NPL2). Eculizumab inhibits the cleavage of C5 into C5a and C5b by C5 convertase, which prevents the generation of the terminal complement complex C5b-9. Both C5a and C5b-9 cause the terminal complement-mediated events that are characteristic of PNH and aHUS (See also PTL3, PTL4, PTL5, and PTL6).

Several reports have described anti-C5 antibodies. For example, PTL7 described an anti-C5 antibody which binds to the alpha chain of C5 but does not bind to C5a, and blocks the activation of C5, while PTL8 described an anti-C5 monoclonal antibody which inhibits C5a formation. On the other hand, PTL9 described an anti-C5 antibody which recognizes the proteolytic site for C5 convertase on the alpha chain of C5, and inhibits the conversion of C5 to C5a and C5b. PTL10 described an anti-C5 antibody which has an affinity constant of at least 1 x10⁷ M⁻¹.

Antibodies (IgGs) bind to neonatal Fc receptor (FcRn), and have long plasma retention times. The binding of IgGs to FcRn is observed only under acidic conditions (e.g. pH 6.0), but it is hardly observed under neutral conditions (e.g. pH 7.4). Typically, IgGs are nonspecifically incorporated into cells via endocytosis, and return to the cell surfaces by binding to endosomal FcRn under the acidic conditions in the endosomes. Then, IgGs dissociate from FcRn under the neutral conditions in plasma. IgGs that have failed to bind to FcRn are degraded in lysosomes. When the FcRn binding ability of an IgG under acidic conditions is eliminated by introducing mutations into its Fc region, the IgG is not recycled from the endosomes into the plasma, leading to marked impairment of the plasma retention of the IgG. To improve the plasma retention of IgGs, a method that enhances their FcRn binding under acidic conditions has been reported. The method is also called “an FcRn-mediated recycling mechanism” hereinafter. When the FcRn binding of an IgG under acidic conditions is improved by introducing an amino acid substitution into its Fc region, the IgG is more efficiently recycled from the endosomes to the plasma, and thereby shows improved plasma retention. Meanwhile, it has also been reported that an IgG with enhanced FcRn binding under neutral conditions does not dissociate from FcRn under the neutral conditions in plasma even when it returns to the cell surface via its binding to FcRn under the acidic conditions in the endosomes, and consequently its plasma retention remains unaltered, or rather, is worsened (See, e.g., NPL3; NPL4; NPL5).

Recently, antibodies that bind to antigens in a pH-dependent manner have been reported (See, e.g., PTL11 and PTL12). The antibodies strongly bind to antigens under the plasma neutral conditions and dissociate from the antigens under the endosomal acidic conditions. After dissociating from the antigens, they become capable once again of binding to antigens when recycled to the plasma via FcRn. Thus, a single antibody molecule can repeatedly bind to multiple antigen molecules. In general, the plasma retention of an antigen is much shorter than that of an antibody, which has the above-mentioned FcRn-mediated recycling mechanism. Therefore, when an antigen is bound to an antibody, the antigen normally shows prolonged plasma retention, resulting in an increase of the plasma concentration of the antigen. On the other hand, it has been reported that the above-described antibodies, which bind to antigens in a pH-dependent manner, eliminate antigens from plasma more rapidly than typical antibodies because they dissociate from the antigens within the endosomes during the FcRn-mediated recycling process. In addition, PTL13 disclosed that antigen elimination from plasma as compared to typical antibodies could be promoted when antibodies that bind to antigens in a pH-dependent manner and form an immune complex comprising two or more antibodies could be promoted. In PTL13, it was suggested that inclusion of two or more Fc regions in such a complex may allow such a complex to be incorporated into cells through binding of antibodies to Fc receptors with an avidity and lead to enhanced elimination of antigens from plasma. PTL14 also described computer modeling analysis showing that an antibody with pH-dependent binding directed against C5 could extend antigen knockdown.

### Citation List

Patent Literature
PTL1U.S. Pat. No. 6,355,245
PTL2U.S. Pat. No. 7,432,356
PTL3WO 2005/074607
PTL4WO 2007/106585
PTL5WO 2008/069889
PTL6WO 2010/054403
PTL7WO 95/29697
PTL8WO 02/30985
PTL9WO 2004/007553
PTL10WO 2010/015608
PTL11WO 2009/125825
PTL12WO 2011/122011
PTL13WO 2013/081143
PTL14WO2011/111007

Non Patent Literature
NPL1Holers et al. (2008) Immunological Reviews 223: 300-316
NPL2Dmytrijuk et al (2008) The Oncologist 13(9): 993-1000
NPL3Yeung et al (2009) J Immunol 182(12): 7663-7671
NPL4Datta-Mannan et al (2007) J Biol Chem 282(3): 1709-1717
NPL5Dall′Acqua et al (2002) J Immunol 169(9): 5171-5180

### SUMMARY OF INVENTION

### Technical Problem

An objective of the invention is to provide a combination of two or more anti-C5 antibodies and methods of using the same.

### Solution to Problem

The invention provides a combination of two or more anti-C5 antibodies and methods of using the same.

In some embodiments, an isolated or purified anti-C5 antibody comprised in a combination of two or more isolated or purified anti-C5 antibodies of the present invention binds to an epitope within the beta chain (SEQ ID NO: 1) or alpha chain (SEQ ID NO: 10) of C5. In some embodiments, an isolated or purified anti-C5 antibody comprised in a combination of two or more isolated or purified anti-C5 antibodies of the present invention binds to an epitope within the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5. In some embodiments, an isolated or purified anti-C5 antibody comprised in a combination of two or more isolated or purified anti-C5 antibodies of the present invention binds to an epitope within a fragment consisting of amino acids 33-124 of the beta chain (SEQ ID NO: 1) or a fragment consisting of amino acids 1-999 of the alpha chain (SEQ ID NO: 10) of C5. In further embodiments, the antibody binds to C5 with a higher affinity at neutral pH than at acidic pH. In further embodiments, the antibody binds to C5 with a higher affinity at higher concentration of calcium than lower concentration of calcium. In another embodiment, an isolated or purified anti-C5 antibody comprised in a combination of two or more isolated or purified anti-C5 antibodies of the present invention binds to the same epitope as any one of the reference antibodies described in Table 2. In another embodiment, an isolated or purified anti-C5 antibody comprised in a combination of two or more isolated or purified anti-C5 antibodies of the present invention compete with any one of the reference antibodies described in Table 2 for binding to C5. Such an isolated or purified anti-C5 antibody of the present invention can modulate, inhibit, block or neutralize a biological function of C5. In some embodiments, an isolated or purified anti-C5 antibody which binds to an epitope selected from any one of i to iii; i the beta chain (SEQ ID NO: 1) or alpha chain (SEQ ID NO: 10) of C5, ii the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5, or iii a fragment consisting of amino acids 33-124 of the beta chain (SEQ ID NO: 1) or a fragment consisting of amino acids 1-999 of the alpha chain (SEQ ID NO: 10) of C5, comprised in a combination of two or more isolated or purified anti-C5 antibodies of the present invention, is a monoclonal antibody. In some embodiments, an isolated or purified anti-C5 antibody which binds to an epitope selected from any one of i to iii; i the beta chain (SEQ ID NO: 1) or alpha chain (SEQ ID NO: 10) of C5, ii the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5, or iii a fragment consisting of amino acids 33-124 of the beta chain (SEQ ID NO: 1) or a fragment consisting of amino acids 1-999 of the alpha chain (SEQ ID NO: 10) of C5, comprised in a combination of two or more isolated or purified anti-C5 antibodies of the present invention, is a human, humanized, or chimeric antibody. In some embodiments, an isolated or purified anti-C5 antibody which binds to an epitope selected from any one of i to iii; i the beta chain (SEQ ID NO: 1) or alpha chain (SEQ ID NO: 10) of C5, ii the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5, or iii a fragment consisting of amino acids 33-124 of the beta chain (SEQ ID NO: 1) or a fragment consisting of amino acids 1-999 of the alpha chain (SEQ ID NO: 10) of C5, comprised in a combination of two or more isolated or purified anti-C5 antibodies the present invention, is a full length IgG1 or IgG4 antibody.

In some embodiments, a combination of two or more isolated or purified anti-C5 antibodies of the present invention can be an isolated or purified multispecific antibody which binds to at least two epitopes within the beta chain (SEQ ID NO: 1) or alpha chain (SEQ ID NO: 10) of C5 that are distinct from each other, wherein the binding sites of the isolated or purified multispecific antibody do not compete with each other for binding to the epitope. In some embodiments, a combination of two or more isolated or purified anti-C5 antibodies of the present invention can be an isolated or purified multispecific antibody which binds to at least of two epitopes within the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5, wherein the binding sites of the isolated or purified multispecific antibody do not compete with each other for binding to the epitope. In some embodiments, a combination of two or more isolated or purified anti-C5 antibodies of the present invention can be an isolated or purified multispecific antibody which binds to at least two epitopes within a fragment consisting of amino acids 33-124 of the beta chain (SEQ ID NO: 1) or a fragment consisting of amino acids 1-999 of the alpha chain (SEQ ID NO: 10) of C5, wherein the binding sites of the isolated or purified multispecific antibody do not compete with each other for binding to the epitope. In further embodiments, a combination of two or more isolated or purified anti-C5 antibodies of the present invention can be an isolated or purified multispecific antibody which binds to at least two epitopes within C5 wherein one or more binding sites of the isolated or purified multispecific antibody bind to C5 with a higher affinity at neutral pH than at acidic pH, and wherein the binding sites of the isolated or purified multispecific antibody do not compete with each other for binding to the epitope. In further embodiments, a combination of two or more isolated or purified anti-C5 antibodies of the present invention can be an isolated or purified multispecific antibody which binds to at least two epitopes within C5 wherein one or more binding sites of the isolated or purified multispecific antibody bind to C5 with a higher affinity at higher concentration of calcium than at lower concentration of calcium, and wherein the binding sites of the isolated or purified multispecific antibody do not compete with each other for binding to the epitope. In another embodiment, a combination of two or more isolated or purified anti-C5 antibodies of the present invention can be an isolated or purified multispecific antibody which binds to at least two epitopes bound by reference antibodies described in Table 2, wherein the binding sites of the isolated or purified multispecific antibody do not compete with each other for binding to the epitope. In another embodiment, a combination of two or more isolated or purified anti-C5 antibodies of the present invention can be an isolated or purified multispecific antibody which competes with at least two reference antibodies described in Table 2 for binding to C5, wherein the binding sites of the isolated or purified multispecific antibody do not compete with each other for binding to the epitope. One or more binding sites of such an isolated or purified multispecific antibody of the present invention can modulate, inhibit, block or neutralize a biological function of C5. In some embodiments, an isolated or purified anti-C5 multispecific antibody of the present invention which binds to at least two epitopes selected from any one of i to iii; i the beta chain (SEQ ID NO: 1) or alpha chain (SEQ ID NO: 10) of C5, ii the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5, or iii a fragment consisting of amino acids 33-124 of the beta chain (SEQ ID NO: 1) or a fragment consisting of amino acids 1-999 of the alpha chain (SEQ ID NO: 10) of C5, wherein the binding sites of the isolated or purified multispecific antibody do not compete with each other for binding to the epitope, is a monoclonal antibody. In some embodiments, an isolated or purified multispecific anti-C5 antibody of the present invention which binds to at least two epitopes selected from any one of i to iii; i the beta chain (SEQ ID NO: 1) or alpha chain (SEQ ID NO: 10) of C5, ii the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5, or iii a fragment consisting of amino acids 33-124 of the beta chain (SEQ ID NO: 1) or a fragment consisting of amino acids 1-999 of the alpha chain (SEQ ID NO: 10) of C5, wherein the binding sites of the isolated or purified multispecific antibody do not compete with each other for binding to the epitope, is a human, humanized, or chimeric antibody. In some embodiments, an isolated or purified anti-C5 multispecific antibody of the present invention which binds to at least two epitopes selected from any one of i to iii; i the beta chain (SEQ ID NO: 1) or alpha chain (SEQ ID NO: 10) of C5, ii the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5, or iii a fragment consisting of amino acids 33-124 of the beta chain (SEQ ID NO: 1) or a fragment consisting of amino acids 1-999 of the alpha chain (SEQ ID NO: 10) of C5, wherein the binding sites of the isolated or purified multispecific antibody do not compete with each other for binding to the epitope, is a full length IgG1 or IgG4 antibody.

In some embodiments, a combination of two or more isolated or purified anti-C5 antibodies of the present invention can be a combination of two or more isolated or purified anti-C5 antibodies, wherein one isolated or purified antibody of the present invention binds to an epitope within the beta chain (SEQ ID NO: 1) or alpha chain (SEQ ID NO: 10) of C5 and wherein the isolated or purified anti-C5 antibodies to be combined do not compete with each other for binding to the epitope. In some embodiments, a combination of two or more isolated or purified anti-C5 antibodies of the present invention can be a combination of two or more isolated or purified anti-C5 antibodies, wherein one isolated or purified antibody binds to an epitope within the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO:11) or C5-C345C/NTR domain (SEQ ID NO:12) of the alpha chain of C5 and wherein the isolated or purified anti-C5 antibodies to be combined do not compete with each other for binding to the epitope. In some embodiments, a combination of two or more isolated or purified anti-C5 antibodies of the present invention can be a combination of two or more isolated or purified anti-C5 antibodies, wherein one isolated or purified antibody binds to an epitope within a fragment consisting of amino acids 33-124 of the beta chain (SEQ ID NO: 1) or a fragment consisting of amino acids 1-999 of the alpha chain (SEQ ID NO: 10) of C5 and wherein the isolated or purified anti-C5 antibodies to be combined do not compete with each other for binding to the epitope. In some embodiments, a combination of two or more isolated or purified anti-C5 antibodies of the present invention can be a combination of two or more isolated or purified anti-C5 antibodies, wherein one isolated or purified antibody binds to an epitope within a fragment consisting of amino acids 33-124 of the beta chain (SEQ ID NO: 1) or a fragment consisting of amino acids 1-999 of alpha chain (SEQ ID NO: 10) of C5 and wherein the isolated or purified anti-C5 antibodies to be combined do not compete with each other for binding to the epitope. In some embodiments, a combination of two or more isolated or purified anti-C5 antibodies of the present invention can be a combination of two or more isolated or purified anti-C5 antibodies which bind to an epitope within the beta chain (SEQ ID NO: 1) or alpha chain (SEQ ID NO: 10) of C5 and wherein the isolated or purified anti-C5 antibodies to be combined do not compete with each other for binding to the epitope. In some embodiments, a combination of two or more isolated or purified anti-C5 antibodies of the present invention can be a combination of two or more isolated or purified anti-C5 antibodies which bind to an epitope within the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5 and wherein the isolated or purified anti-C5 antibodies to be combined do not compete with each other for binding to the epitope. In some embodiments, a combination of two or more isolated or purified anti-C5 antibodies of the present invention can be a combination of two or more isolated or purified anti-C5 antibodies which bind to an epitope within a fragment consisting of amino acids 33-124 of the beta chain (SEQ ID NO: 1) or a fragment consisting of amino acids 1-999 of the alpha chain (SEQ ID NO: 10) of C5 and wherein the isolated or purified anti-C5 antibodies to be combined do not compete with each other for binding to the epitope. In further embodiments, a combination of two or more isolated or purified anti-C5 antibodies of the present invention can comprise one or more of the isolated or purified anti-C5 antibodies to be combined which bind to C5 with a higher affinity at neutral pH than at acidic pH, wherein the isolated or purified anti-C5 antibodies to be combined do not compete with each other for binding to the epitope. In further embodiments, a combination of two or more isolated or purified anti-C5 antibodies of the present invention can comprise one or more of the isolated or purified anti-C5 antibodies to be combined which bind to C5 with a higher affinity at higher concentration of calcium than at lower concentration of calcium, wherein the isolated or purified anti-C5 antibodies to be combined do not compete with each other for binding to the epitope. In another embodiment, a combination of two or more isolated or purified anti-C5 antibodies of the present invention can be a combination of two or more isolated or purified anti-C5 antibodies, wherein one or more antibodies to be combined bind to epitopes bound by reference antibodies described in Table 2, wherein the isolated or purified anti-C5 antibodies to be combined do not compete with each other for binding to the epitope. In another embodiment, a combination of two or more isolated or purified anti-C5 antibodies of the present invention can be a combination of two or more isolated or purified anti-C5 antibodies which bind to two or more epitopes bound by reference antibodies described in Table 2, wherein the isolated or purified anti-C5 antibodies to be combined do not compete with each other for binding to the epitope. In another embodiment, a combination of two or more isolated or purified anti-C5 antibodies of the present invention can be a combination of two or more isolated or purified antibodies, wherein one or more antibodies to be combined compete with reference antibodies described in Table 2 for binding to C5, wherein the isolated or purified anti-C5 antibodies to be combined do not compete with each other for binding to the epitope. In another embodiment, a combination of two or more isolated or purified anti-C5 antibodies of the present invention can be a combination of two or more isolated or purified antibodies which compete with at least two reference antibodies described in Table 2 for binding to C5, wherein the isolated or purified anti-C5 antibodies to be combined do not compete with each other for binding to the epitope. One or more of the isolated or purified anti-C5 antibodies comprised in such a combination of at least two isolated or purified antibodies of the present invention can modulate, inhibit, block or neutralize a biological function of C5.

In some embodiments, one or more of the isolated or purified antibodies comprised in the combination of this invention, wherein the isolated or purified anti-C5 antibodies to be combined do not compete with each other for binding to the epitope and wherein one or more of the epitopes is selected from any one of i to iii; i the beta chain (SEQ ID NO: 1) or alpha chain (SEQ ID NO: 10) of C5, ii the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5, or iii a fragment consisting of amino acids 33-124 of the beta chain (SEQ ID NO: 1) or a fragment consisting of amino acids 1-999 of the alpha chain (SEQ ID NO: 10) of C5, are monoclonal antibodies. In some embodiments, one or more of the isolated or purified antibodies comprised in the combination of this invention, wherein the isolated or purified anti-C5 antibodies to be combined do not compete with each other for binding to the epitope and wherein one or more of the epitopes are selected from any one of i to iii; i the beta chain (SEQ ID NO: 1) or alpha chain (SEQ ID NO: 10) of C5, ii the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5, or iii a fragment consisting of amino acids 33-124 of the beta chain (SEQ ID NO: 1) or a fragment consisting of amino acids 1-999 of the alpha chain (SEQ ID NO: 10) of C5, are human, humanized, or chimeric antibodies, or a combination thereof. In some embodiments, isolated or purified antibodies comprised in the combination of this invention, wherein the isolated or purified anti-C5 antibodies to be combined do not compete with each other for binding to the epitope and wherein one or more of the epitopes are selected from any one of i to iii; i the beta chain (SEQ ID NO: 1) or alpha chain (SEQ ID NO: 10) of C5, ii the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5, or iii a fragment consisting of amino acids 33-124 of the beta chain (SEQ ID NO: 1) or a fragment consisting of amino acids 1-999 of the alpha chain (SEQ ID NO: 10) of C5, are full length IgG1 or IgG4 antibodies. In further embodiments, a combination of two or more isolated or purified anti-C5 antibodies of the present invention can be a combination of at least two isolated or purified anti-C5 antibodies which bind to C5 with a higher affinity at neutral pH than at acidic pH, wherein the isolated or purified anti-C5 antibodies to be combined do not compete with each other for binding to the epitope. In further embodiments, a combination of two or more isolated or purified anti-C5 antibodies of the present invention can comprise one or more of the isolated or purified anti-C5 antibodies to be combined which bind to C5 with a higher affinity at higher concentration of calcium than at lower concentration of calcium, wherein the isolated or purified anti-C5 antibodies to be combined do not compete with each other for binding to the epitope. In another embodiment, a combination of two or more isolated or purified anti-C5 antibodies of the present invention can be a combination of two or more isolated or purified anti-C5 antibodies which bind to two or more epitopes bound by reference antibodies described in Table 2, wherein the isolated or purified anti-C5 antibodies to be combined do not compete with each other for binding to the epitope. In another embodiment, a combination of two or more isolated or purified anti-C5 antibodies of the present invention can be a combination of two or more isolated or purified antibodies which compete with at least two reference antibodies described in Table 2 for binding to C5, wherein the isolated or purified anti-C5 antibodies to be combined do not compete with each other for binding to the epitope. One or more of the isolated or purified anti-C5 antibodies comprised in such a combination of at least two isolated or purified antibodies of the present invention can modulate, inhibit, block or neutralize a biological function of C5.

The invention also provides a pharmaceutical formulation comprising a combination of two or more anti-C5 antibodies of the present invention and a pharmaceutically acceptable carrier.

A combination of two or more anti-C5 antibodies of the present invention may be for use as a medicament. A combination of two or more anti-C5 antibodies of the present invention may be for use in treating a complement-mediated disease or condition which involves excessive or uncontrolled activation of C5. A combination of two or more anti-C5 antibodies of the present invention may be for use in enhancing the clearance of C5 from plasma.

A combination of two or more anti-C5 antibodies of the present invention may be used in the manufacture of a medicament. In some embodiments, the medicament is for treatment of a complement-mediated disease or condition which involves excessive or uncontrolled activation of C5. In some embodiments, the medicament is for enhancing the clearance of C5 from plasma.

The invention also provides a method of treating an individual having a complement-mediated disease or condition which involves excessive or uncontrolled activation of C5. In some embodiments, the method comprises administering to the individual an effective amount of a combination of two or more anti-C5 antibodies of the present invention. The invention also provides a method of enhancing the clearance of C5 from plasma in an individual. In some embodiments, the method comprises administering to the individual an effective amount of a combination of two or more anti-C5 antibodies of the present invention to enhance the clearance of C5 from plasma.

Specifically, the present invention relates to:

1 A combination of two or more isolated or purified anti-C5 antibodies, wherein the isolated or purified anti-C5 antibodies bind to an epitope within the beta chain (SEQ ID NO: 1) or alpha chain (SEQ ID NO: 10) of C5 and wherein the isolated or purified anti-C5 antibodies to be combined do not compete with each other for binding to the epitope.

2 The combination according to 1, wherein the epitope is selected from an epitope within the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or an epitope within the anaphylatoxin domain (SEQ ID NO: 11) or C5-C345C/NTR domain (SEQ ID NO: 12) of the alpha chain of C5.

3 The combination according to 1 or 2, wherein the epitope is selected from within a fragment consisting of amino acids 33-124 of the beta chain (SEQ ID NO: 1) or a fragment consisting of amino acids 1-999 of the alpha chain (SEQ ID NO: 10) of C5.

4 The combination according to any one of 1 to 3, wherein one or more of the anti-C5 antibodies bind to C5 with a higher affinity at neutral pH than at acidic pH.

5 The combination according to any one of 1 to 4, wherein one or more of the isolated or purified anti-C5 antibodies bind to the same epitope as any one of reference antibodies described in Table 2.

6 The combination according to any one of 1 to 5, wherein one or more of the isolated or purified anti-C5 antibodies compete with any one of reference antibodies described in Table 2 for binding to C5.

7 The combination according to any one of 1 to 5, wherein one or more of the isolated or purified anti-C5 antibodies comprise 6 HVRs of any one of antibodies described in Table 2.

8 The combination according to any one of 1 to 7, wherein one or more of the isolated or purified anti-C5 antibodies modulate, inhibit, block or neutralize a biological function of C5.

9 The combination according to any one of 1 to 8, wherein one or more of the isolated or purified anti-C5 antibodies are a monoclonal antibody.

10 The combination according to any one of 1 to 9, wherein one or more of the isolated or purified anti-C5 antibodies are a human, humanized, or chimeric antibody.

11 The combination according to any one of 1 to 10, wherein one or more of the isolated or purified anti-C5 antibodies are a full length IgG1 or IgG4 antibody.

12 The combination according to any one of 1 to 11, wherein the combination of isolated or purified anti-C5 antibodies is an isolated or purified multispecific antibody.

13 A pharmaceutical formulation comprising the combination of any one of 1 to 12 and a pharmaceutically acceptable carrier.

14 The combination of any one of 1 to 11 for use as a medicament.

15 The combination of any one of 1 to 11 for use in treating a complement-mediated disease or condition which involves excessive or uncontrolled activation of C5.

16 The combination of any one of 1 to 11 for use in enhancing the clearance of C5 from plasma.

17 Use of the combination of any one of 1 to 11 in the manufacture of a medicament for treatment of a complement-mediated disease or condition which involves excessive or uncontrolled activation of C5.

18 Use of the combination of any one of 1 to 11 in the manufacture of a medicament for enhancing the clearance of C5 from plasma.

19 A method of treating an individual having a complement-mediated disease or condition which involves excessive or uncontrolled activation of C5, the method comprising administering to the individual an effective amount of the combination of any one of 1 to 11.

20 A method of enhancing the clearance of C5 from plasma in an individual comprising administering to the individual an effective amount of the combination of any one of 1 to 11 to enhance the clearance of C5 from plasma.

### BRIEF DESCRIPTION OF DRAWINGS

FIG. 1-1 shows Octet sensorgrams of selected 25 twenty five pH-dependent and/or calcium-dependent antigen binding clones.

FIGS. 1-2 is continuation of FIG. 1-1.

FIGS. 2-1 shows comparison of mFcRn binding between immune complexes comprising anti-C5 bispecific antibodies and anti-C5 monoclonal antibodies.

FIG. 2-2 is continuation of FIGS. 2-1.

FIG. 3A shows sequence comparison of HVRs between two light chains comprised in anti-C5 bispecific antibodies. Positions of residues are designated according to Kabat numbering.

FIG. 3B is continuation of FIG. 3A.

FIG. 4 shows Biacore binding sensorgrams of clones 20 and 18 comprising parent or common light chain to C5.

FIG. 5 shows time profiles of plasma concentration of total C5 in human FcRn transgenic mice after injection of anti-C5 bispecific antibodies.

FIG. 6 shows adjusted Biacore binding sensorgrams of 20//18 variants to C5. Solid lines show association with human C5 and dissociation from human C5 at pH 7.4. Dashed lines show association with human C5 at pH 7.4 and dissociation from human C5 at pH 5.8.

FIG. 7 shows time profiles of plasma concentration of total C5 in cynomolgus monkey after injection of Fc variants of optimized 20//18.

### DESCRIPTION OF EMBODIMENTS

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F.M. Ausubel, et al. eds., (2003)); the series Methods in Enzymology (Academic Press, Inc.): PCR 2: A Practical Approach (M.J. MacPherson, B.D. Hames and G.R. Taylor eds. (1995)), Harlow and Lane, eds. (1988) Antibodies, A Laboratory Manual, and Animal Cell Culture (R.I. Freshney, ed. (1987)); Oligonucleotide Synthesis (M.J. Gait, ed., 1984); Methods in Molecular Biology, Humana Press; Cell Biology: A Laboratory Notebook (J.E. Cellis, ed., 1998) Academic Press; Animal Cell Culture (R.I. Freshney), ed., 1987); Introduction to Cell and Tissue Culture (J. P. Mather and P.E. Roberts, 1998) Plenum Press; Cell and Tissue Culture: Laboratory Procedures (A. Doyle, J.B. Griffiths, and D.G. Newell, eds., 1993-8) J. Wiley and Sons; Handbook of Experimental Immunology (D.M. Weir and C.C. Blackwell, eds.); Gene Transfer Vectors for Mammalian Cells (J.M. Miller and M.P. Calos, eds., 1987); PCR: The Polymerase Chain Reaction, (Mullis et al., eds., 1994); Current Protocols in Immunology (J.E. Coligan et al., eds., 1991); Short Protocols in Molecular Biology (Wiley and Sons, 1999); Immunobiology (C.A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V.T. DeVita et al., eds., J.B. Lippincott Company, 1993).

### I. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. All references cited herein, including patent applications and publications, are incorporated by reference in their entirety.

For purposes of interpreting this specification, the following definitions will apply and whenever appropriate, terms used in the singular will also include the plural and vice versa. It is to be understood that the terminology used herein is for the purpose of describing particular embodiments only, and is not intended to be limiting. In the event that any definition set forth below conflicts with any document incorporated herein by reference, the definition set forth below shall control.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-C5 antibody” and “an antibody that binds to C5” refer to an antibody that is capable of binding C5 with sufficient affinity such that the antibody is useful as a diagnostic and/or therapeutic agent in targeting C5. In one embodiment, the extent of binding of an anti-C5 antibody to an unrelated, non-C5 protein is less than about 10% of the binding of the antibody to C5 as measured, e.g., by a radioimmunoassay (RIA). In certain embodiments, an antibody that binds to C5 has a dissociation constant (Kd) of ≤ 1 µM, ≤ 100 nM, ≤ 10 nM, ≤ 1 nM, ≤ 0.1 nM, ≤ 0.01 nM, or ≤ 0.001 nM\ (e.g. 10⁻⁸ M or less, e.g. from 10⁻⁸ M to 10⁻¹³ M, e.g., from 10⁻⁹ M to 10⁻¹³ M). In certain embodiments, an anti-C5 antibody binds to an epitope of C5 that is conserved among C5 from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay, and/or conversely, the reference antibody blocks binding of the antibody to its antigen in a competition assay. An exemplary competition assay is provided herein.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called alpha, delta, epsilon, gamma, and mu, respectively.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, 1¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

The term “epitope” includes any determinant capable of being bound by an antibody. An epitope is a region of an antigen that is bound by an antibody that targets that antigen, and includes specific amino acids that directly contact the antibody. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of proteins and/or macromolecules.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

An “isolated” or “purified” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For review of methods for assessment of antibody purity, see, e.g., Flatman et al., J. Chromatogr. B 848:79-87 (2007).

An “isolated” or “purified” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-C5 antibody” or “purified nucleic acid encoding an anti-C5 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (kappa) and lambda (lambda), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

$$100 \text{times the fraction} \text{X} / \text{Y}$$

where X is the number of amino acid residues scored as identical matches by the sequence alignment program ALIGN-2 in that program’s alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject., A pharmaceutically acceptable carrier includes, but is not limited to, a buffer, excipient, stabilizer, or preservative.

The term “C5”, as used herein, refers to any native C5 from any vertebrate source, including mammals such as primates (e.g. humans and monkeys) and rodents (e.g., mice and rats), unless otherwise indicated. The term encompasses “full-length,” unprocessed C5 as well as any form of C5 that results from processing in the cell. The term also encompasses naturally occurring variants of C5, e.g., splice variants or allelic variants. The amino acid sequence of an exemplary human C5 is shown in SEQ ID NO: 13. The amino acid sequence of an exemplary beta chain of human C5 is shown in SEQ ID NO: 1. The amino acid sequence of an exemplary MG1, MG2, MG3, MG4, MG5, MG6, MG1-MG2 and MG3-MG6 domain of the beta chain of human C5 is shown in SEQ ID NOs: 2, 3, 4, 5, 6, 7, 8 and 9, respectively. The amino acid sequence of an exemplary alpha chain of human C5 is shown in SEQ ID NO: 10. The amino acid sequence of an exemplary anaphylatoxin domain and C5-C345C/NTR domain of the alpha chain of human C5 is shown in SEQ ID NO: 11 and 12, respectively. The amino acid sequences of an exemplary cynomolgus monkey and murine C5 are shown in SEQ ID NO: 14 and 62, respectively.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. Compositions and Methods

In one aspect, the invention is based, in part, on anti-C5 antibodies and uses thereof. In certain embodiments, antibodies that bind to C5 are provided. Antibodies of the invention are useful, e.g., for the diagnosis or treatment of a complement-mediated disease or condition which involves excessive or uncontrolled activation of C5.

### A. Exemplary Anti-C5 Antibodies

In one aspect, the invention provides isolated antibodies that bind to C5. In certain embodiments, an anti-C5 antibody of the present invention binds to an epitope within the beta chain (SEQ ID NO: 1) or alpha chain (SEQ ID NO: 10) of C5. In certain embodiments, the anti-C5 antibody binds to an epitope within the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5. In certain embodiments, the anti-C5 antibody binds to an epitope within a fragment consisting of amino acids 19-180 of the beta chain of C5 or a fragment consisting of amino acids 1-999 of the alpha chain (SEQ ID NO: 10) of C5.

In another aspect, the invention provides anti-C5 antibodies that exhibit pH-dependent binding characteristics or calcium-dependent binding characteristics. As used herein, the expression “pH-dependent binding” means that the antibody exhibits “reduced binding to C5 at acidic pH as compared to its binding at neutral pH” (for purposes of the present disclosure, both expressions may be used interchangeably). For example, antibodies “with pH-dependent binding characteristics” include antibodies that bind to C5 with higher affinity at neutral pH than at acidic pH. In certain embodiments, the antibodies of the present invention bind to C5 with at least 2, 3, 5, 10, 15, 20, 25, 30, 35, 40, 45, 50, 55, 60, 65, 70, 75, 80, 85, 90, 95, 100, 200, 400, 1000, 10000, or more times higher affinity at neutral pH than at acidic pH. As used herein, the expression “calcium-dependent binding or calcium concentration binding” means that the antibody exhibits “reduced binding to C5 at lower concentration of calcium as compared to its binding at higher concentration of calcium” (for purposes of the present disclosure, both expressions may be used interchangeably). For example, antibodies “with calcium-dependent binding characteristics” include antibodies that bind to C5 with higher affinity at higher concentration of calcium than at lower concentration of calcium. In certain embodiments, the antibodies of the present invention bind to C5 with at least 2, 3, 5, 10, 15, 20, 25, 30, 35, 40, 45, 50, 55, 60, 65, 70, 75, 80, 85, 90, 95, 100, 200, 400, 1000, 10000, or more times higher affinity at higher concentration of calcium than at lower concentration of calcium.

The “affinity” of an antibody for C5, for purposes of the present disclosure, is expressed in terms of the KD of the antibody. The KD of an antibody refers to the equilibrium dissociation constant of an antibody-antigen interaction. The greater the KD value is for an antibody binding to its antigen, the weaker its binding affinity is for that particular antigen. Accordingly, as used herein, the expression “higher affinity at neutral pH than at acidic pH” (or the equivalent expression “pH-dependent binding”) means that the KD for the antibody binding to C5 at acidic pH is greater than the KD for the antibody binding to C5 at neutral pH. For example, in the context of the present invention, an antibody is considered to bind to C5 with a higher affinity at neutral pH than at acidic pH if the KD of the antibody binding to C5 at acidic pH is at least 2 times greater than the KD of the antibody binding to C5 at neutral pH. Thus, the present invention includes antibodies that bind to C5 at acidic pH with a KD that is at least 2, 3, 5, 10, 15, 20, 25, 30, 35, 40, 45, 50, 55, 60, 65, 70, 75, 80, 85, 90, 95, 100, 200, 400, 1000, 10000, or more times greater than the KD of the antibody binding to C5 at neutral pH. Accordingly, as used herein, the expression “higher affinity at higher concentration of calcium than at lower concentration of calcium” (or the equivalent expression “calcium-dependent binding or calcium concentration-dependent binding”) means that the KD for the antibody binding to C5 at lower concentration of calcium is greater than the KD for the antibody binding to C5 at higher concentration of calcium. For example, in the context of the present invention, an antibody is considered to bind to C5 with a higher affinity at higher concentration of calcium than at lower concentration of calcium if the KD of the antibody binding to C5 at lower concentration of calcium is at least 2 times greater than the KD of the antibody binding to C5 at higher concentration of calcium. Thus, the present invention includes antibodies that bind to C5 at lower concentration of calcium with a KD that is at least 2, 3, 5, 10, 15, 20, 25, 30, 35, 40, 45, 50, 55, 60, 65, 70, 75, 80, 85, 90, 95, 100, 200, 400, 1000, 10000, or more times greater than the KD of the antibody binding to C5 at higher concentration of calcium.

The binding properties of an antibody for a particular antigen may also be expressed in terms of the kd of the antibody. The kd of an antibody refers to the dissociation rate constant of the antibody with respect to a particular antigen and is expressed in terms of reciprocal seconds (i.e., sec⁻¹). An increase in kd value signifies weaker binding of an antibody to its antigen. The present invention therefore includes antibodies that bind to C5 with a higher kd value at acidic pH than at neutral pH. The present invention includes antibodies that bind to C5 at acidic pH with a kd that is at least 2, 3, 5, 10, 15, 20, 25, 30, 35, 40, 45, 50, 55, 60, 65, 70, 75, 80, 85, 90, 95, 100, 200, 400, 1000, 10000, or more times greater than the kd of the antibody binding to C5 at neutral pH. The present invention therefore includes antibodies that bind to C5 with a higher kd value at lower concentration of calcium than at higher concentration of calcium.

In certain instances, a “reduced binding to C5 at acidic pH as compared to its binding at neutral pH” is expressed in terms of the ratio of the KD value of the antibody binding to C5 at acidic pH to the KD value of the antibody binding to C5 at neutral pH (or vice versa). For example, an antibody may be regarded as exhibiting “reduced binding to C5 at acidic pH as compared to its binding at neutral pH”, for purposes of the present invention, if the antibody exhibits an acidic/neutral KD ratio of 2 or greater. In certain exemplary embodiments, the acidic/neutral KD ratio for an antibody of the present invention can be 2, 3, 5, 10, 15, 20, 25, 30, 35, 40, 45, 50, 55, 60, 65, 70, 75, 80, 85, 90, 95, 100, 200, 400, 1000, 10000, or greater.

In certain instances, a “reduced binding to C5 at lower concentration of calcium as compared to its binding at higher concentration of calcium” is expressed in terms of the ratio of the KD value of the antibody binding to C5 at lower concentration of calcium to the KD value of the antibody binding to C5 at higher concentration of calcium (or vice versa). For example, an antibody may be regarded as exhibiting “reduced binding to C5 at lower concentration of calcium as compared to its binding at higher concentration of calcium”, for purposes of the present invention, if the antibody exhibits a lower concentration of calcium/higher concentration of calcium KD ratio of 2 or greater. In certain exemplary embodiments, the lower concentration of calcium/higher concentration of calcium KD ratio for an antibody of the present invention can be 2, 3, 5, 10, 15, 20, 25, 30, 35, 40, 45, 50, 55, 60, 65, 70, 75, 80, 85, 90, 95, 100, 200, 400, 1000, 10000, or greater.

In certain instances, a “reduced binding to C5 at acidic pH as compared to its binding at neutral pH” is expressed in terms of the ratio of the kd value of the antibody binding to C5 at acidic pH to the kd value of the antibody binding to C5 at neutral pH (or vice versa). For example, an antibody may be regarded as exhibiting “reduced binding to C5 at acidic pH as compared to its binding at neutral pH”, for purposes of the present invention, if the antibody exhibits an acidic/neutral kd ratio of 2 or greater. In certain exemplary embodiments, the acidic/neutral kd ratio for an antibody of the present invention can be 2, 3, 5, 10, 15, 20, 25, 30, 35, 40, 45, 50, 55, 60, 65, 70, 75, 80, 85, 90, 95, 100, 200, 400, 1000, 10000, or greater.

In certain instances, a “reduced binding to C5 at lower concentration of calcium as compared to its binding at higher concentration of calcium” is expressed in terms of the ratio of the kd value of the antibody binding to C5 at lower concentration of calcium to the kd value of the antibody binding to C5 at higher concentration of calcium (or vice versa). For example, an antibody may be regarded as exhibiting “reduced binding to C5 at lower concentration of calcium as compared to its binding at higher concentration of calcium”, for purposes of the present invention, if the antibody exhibits a lower concentration of calcium/higher concentration of calcium kd ratio of 2 or greater. In certain exemplary embodiments, the lower concentration of calcium/higher concentration of calcium kd ratio for an antibody of the present invention can be 2, 3, 5, 10, 15, 20, 25, 30, 35, 40, 45, 50, 55, 60, 65, 70, 75, 80, 85, 90, 95, 100, 200, 400, 1000, 10000, or greater.

As used herein, the expression “acidic pH” means a pH of 4.0 to 6.5. The expression “acidic pH” includes pH values of 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, and 6.5. As used herein, the expression “lower concentration of calcium” means a calcium concentration of 0.1 micro M to 30 micro M. The expression “lower concentration of calcium” includes calcium concentrations of 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 21, 22, 23, 24, 25, 26, 27, 28, 29, and 30 micro M.

As used herein, the expression “neutral pH” means a pH of 6.7 to about 10.0. The expression “neutral pH” includes pH values of 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0. As used herein, the expression “higher concentration of calcium” means a calcium concentration of 0.1 mM to about 10 mM. The expression “higher concentration of calcium” includes calcium concentrations of 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, and 10.0 \.mM.

KD values, and kd values, as expressed herein, may be determined using a surface plasmon resonance-based biosensor to characterize antibody-antigen interactions. (See, e.g., Example 3, herein). KD values, and kd values can be determined at 25° C. or 37° C.

It has been discovered in the present invention that a combination of two or more isolated or purified anti-C5 antibodies wherein one isolated or purified anti-C5 antibody binds to an epitope within the beta chain (SEQ ID NO: 1) or alpha chain (SEQ ID NO: 10) of C5 and wherein the isolated or purified anti-C5 antibodies to be combined do not compete with each other for binding to the epitope and optionally wherein such at least one isolated and purified anti-C5 antibodies exhibit pH-dependent or calcium concentration-dependent binding characteristics, eliminates antigens e.g. C5 from plasma when such a combination is administered to a subject. Without being restricted to a particular theory, it can be speculated that a combination of two or more anti-C5 antibodies may form a complex including two or more antigens e.g. C5 and two or more Fc regions comprised in such anti-C5 antibodies. Inclusion of two or more Fc regions in such a complex may allow such a complex to be incorporated into cells through binding of antibodies to Fc receptors with an avidity and lead to enhanced elimination of antigens e.g. C5 from plasma.

In certain embodiments, one or more anti-C5 antibodies comprised in the combination of the present invention binds to C5 from more than one species. In further embodiments, the anti-C5 antibodies bind to C5 from human and non-human animal. In further embodiments, the anti-C5 antibodies bind to C5 from human and monkey (e.g. cynomolgus, rhesus macaque, marmoset, chimpanzee, or baboon).

In one aspect, the invention provides a combination of two or more anti-C5 antibodies, wherein one or more of the antibodies to be combined inhibit activation of C5. In certain embodiments, anti-C5 antibodies are provided which prevent the cleavage of C5 to form C5a and C5b, thus preventing the generation of anaphylatoxic activity associated with C5a, as well as preventing the assembly of the C5b-9 membrane attack complex (MAC) associated with C5b. In certain embodiments, anti-C5 antibodies are provided which block the conversion of C5 into C5a and C5b by C5 convertase. In certain embodiments, anti-C5 antibodies are provided which block access of the C5 convertase to the cleavage site on C5. In certain embodiments, anti-C5 antibodies are provided which block hemolytic activity caused by the activation of C5. In further embodiments, anti-C5 antibodies of the present invention inhibit the activation of C5 via classical pathway and/or alternative pathway.

In one aspect, the invention provides a combination of two or more anti C5 antibodies, wherein one or more of the anti-C5 antibodies comprises at least one, two, three, four, five, or six HVRs selected from (a) HVR-H1 comprising the amino acid sequence of any one of SEQ ID NOs: 63-66; (b) HVR-H2 comprising the amino acid sequence of any one of SEQ ID NOs: 67-71; (c) HVR-H3 comprising the amino acid sequence of any one of SEQ ID NOs: 72-78; (d) HVR-L1 comprising the amino acid sequence of any one of SEQ ID NOs: 36-37; (e) HVR-L2 comprising the amino acid sequence of any one of SEQ ID NOs: 38-41; and (f) HVR-L3 comprising the amino acid sequence of any one of SEQ ID NOs: 42-48.

In one aspect, the invention provides a combination of two or more anti-C5 antibodies, wherein one or more anti-C5 antibodies comprises at least one, at least two, or all three VH HVR sequences selected from (a) HVR-H1 comprising the amino acid sequence of any one of SEQ ID NOs: 63-66; (b) HVR-H2 comprising the amino acid sequence of any one of SEQ ID NOs: 67-71; and (c) HVR-H3 comprising the amino acid sequence of any one of SEQ ID NOs: 72-78. In one embodiment, the antibody comprises HVR-H3 comprising the amino acid sequence of any one of SEQ ID NOs: 72-78. In another embodiment, the antibody comprises HVR-H3 comprising the amino acid sequence of any one of SEQ ID NOs: 72-78 and HVR-L3 comprising the amino acid sequence of any one of SEQ ID NOs: 42-48. In a further embodiment, the antibody comprises HVR-H3 comprising the amino acid sequence of any one of SEQ ID NOs: 72-78, HVR-L3 comprising the amino acid sequence of any one of SEQ ID NOs: 42-48, and HVR-H2 comprising the amino acid sequence of any one of SEQ ID NOs: 67-71. In a further embodiment, the antibody comprises (a) HVR-H1 comprising the amino acid sequence of any one of SEQ ID NOs: 63-66; (b) HVR-H2 comprising the amino acid sequence of any one of SEQ ID NOs: 67-71; and (c) HVR-H3 comprising the amino acid sequence of any one of SEQ ID NOs: 73-78.

In another aspect, the invention provides a combination of two or more anti-C5 antibodies, wherein one or more anti-C5 antibodies comprises at least one, at least two, or all three VL HVR sequences selected from (a) HVR-L1 comprising the amino acid sequence of any one of SEQ ID NOs: 36-37; (b) HVR-L2 comprising the amino acid sequence of any one of SEQ ID NOs: 38-41; and (c) HVR-L3 comprising the amino acid sequence of any one of SEQ ID NOs: 42-48. In one embodiment, the antibody comprises (a) HVR-L1 comprising the amino acid sequence of any one of SEQ ID NOs: 36-37; (b) HVR-L2 comprising the amino acid sequence of any one of SEQ ID NOs: 38-41; and (c) HVR-L3 comprising the amino acid sequence of any one of SEQ ID NOs: 42-48.

In another aspect, an antibody comprised in the combination of the invention comprises (a) a VH domain comprising at least one, at least two, or all three VH HVR sequences selected from (i) HVR-H1 comprising the amino acid sequence of any one of SEQ ID NOs: 63-66, (ii) HVR-H2 comprising the amino acid sequence of any one of SEQ ID NOs: 67-71, and (iii) HVR-H3 comprising the amino acid sequence of any one of SEQ ID NOs: 72-78; and (b) a VL domain comprising at least one, at least two, or all three VL HVR sequences selected from (i) HVR-L1 comprising the amino acid sequence of any one of SEQ ID NOs: 36-37, (ii) HVR-L2 comprising the amino acid sequence of any one of SEQ ID NOs: 38-41, and (c) HVR-L3 comprising the amino acid sequence of any one of SEQ ID NOs: 42-48.

In another aspect, the invention provides a combination of two or more anti-C5 antibodies, wherein one or more anti-C5 antibodies comprises (a) HVR-H1 comprising the amino acid sequence of any one of SEQ ID NOs: 63-66; (b) HVR-H2 comprising the amino acid sequence of any one of SEQ ID NOs: 67-71; (c) HVR-H3 comprising the amino acid sequence of any one of SEQ ID NOs: 72-78; (d) HVR-L1 comprising the amino acid sequence of any one of SEQ ID NOs: 36-37; (e) HVR-L2 comprising the amino acid sequence of any one of SEQ ID NOs: 38-41; and (f) HVR-L3 comprising the amino acid sequence of any one of SEQ ID NOs: 42-48.

In another aspect, one or more anti-C5 antibodies comprised in the combination of the invention comprises a heavy chain variable domain (VH) sequence having at least 90%, 91%, 92%, 93%, 94%, 95%, 96%, 97%, 98%, 99%, or 100% sequence identity to the amino acid sequence of any one of SEQ ID NOs: 15, 17, 19, 21, 23, 25, 27, 29, 31, 52 and 54. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C5 antibody comprising that sequence retains the ability to bind to C5. In certain embodiments, a total of 1 to 10 amino acids have been substituted, inserted and/or deleted in any one of SEQ ID NOs: 15, 17, 19, 21, 23, 25, 27, 29, 31, 52 and 54. In certain embodiments, substitutions, insertions, or deletions occur in regions outside the HVRs (i.e., in the FRs). Optionally, the anti-C5 antibody comprises the VH sequence in any one of SEQ ID NOs: 15, 17, 19, 21, 23, 25, 27, 29, 31, 52 and 54, including post-translational modifications of that sequence. In a particular embodiment, the VH comprises one, two or three HVRs selected from: (a) HVR-H1 comprising the amino acid sequence of any one of SEQ ID NOs: 63-66, (b) HVR-H2 comprising the amino acid sequence of any one of SEQ ID NOs: 67-71, and (c) HVR-H3 comprising the amino acid sequence of any one of SEQ ID NOs: 72-77.

In another aspect, a combination of two or more anti-C5 antibodies is provided, wherein one or more antibodies comprise a light chain variable domain (VL) having at least 90%, 91%, 92%, 93%, 94%, 95%, 96%, 97%, 98%, 99%, or 100% sequence identity to the amino acid sequence of any one of SEQ ID NOs: 16, 18, 20, 22, 24, 26, 28, 30, 32, 35 and 53. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-C5 antibody comprising that sequence retains the ability to bind to C5. In certain embodiments, a total of 1 to 10 amino acids have been substituted, inserted and/or deleted in any one of SEQ ID NOs: 16, 18, 20, 22, 24, 26, 28, 30, 32, 35 and 53. In certain embodiments, the substitutions, insertions, or deletions occur in regions outside the HVRs (i.e., in the FRs). Optionally, the anti-C5 antibody comprises the VL sequence in any one of SEQ ID NOs: 16, 18, 20, 22, 24, 26, 28, 30, 32, 35 and 53, including post-translational modifications of that sequence. In a particular embodiment, the VL comprises one, two or three HVRs selected from (a) HVR-L1 comprising the amino acid sequence of any one of SEQ ID NOs: 36-37; (b) HVR-L2 comprising the amino acid sequence of any one of SEQ ID NOs: 38-41; and (c) HVR-L3 comprising the amino acid sequence of any one of SEQ ID NOs: 42-48.

In another aspect, a combination of two or more anti-C5 antibodies is provided, wherein one or more antibodies comprise a VH as in any of the embodiments provided above, and a VL as in any of the embodiments provided above. In one embodiment, the antibody comprises the VH and VL sequences in any one of SEQ ID NOs: 15, 17, 19, 21, 23, 25, 27, 29, 31, 52 and 54 and any one of SEQ ID NOs: 16, 18, 20, 22, 24, 26, 28, 30, 32, 35 and 53, respectively, including post-translational modifications of those sequences.

In another aspect, the invention provides a combination of two or more anti-C5 antibodies, wherein one or more antibodies to be combined bind to the same epitope as an anti-C5 antibody provided herein. For example, in certain embodiments, an antibody is provided that binds to the same epitope as an antibody described in Table 2. As demonstrated by the working examples below, all the anti-C5 antibodies described in Table 2 are grouped into the same epitope bin of C5 and exhibit pH-dependent binding characteristics.

In a further aspect of the invention, an anti-C5 antibody according to any of the above embodiments is a monoclonal antibody, including a chimeric, humanized or human antibody. In one embodiment, an anti-C5 antibody is an antibody fragment, e.g., a Fv, Fab, Fab′, scFv, diabody, or F(ab′)₂ fragment. In another embodiment, the antibody is a full length antibody, e.g., an intact IgG1 or IgG4 antibody or other antibody class or isotype as defined herein.

In a further aspect, an anti-C5 antibody according to any of the above embodiments may incorporate any of the features, singly or in combination, as described in Sections 1-7 below:

### 1. Antibody Affinity

In certain embodiments, an antibody provided herein has a dissociation constant (Kd) of ≤ 1 µM, ≤ 100 nM, ≤ 10 nM, ≤ 1 nM, ≤ 0.1 nM, ≤ 0.01 nM, or ≤ 0.001 nM (e.g. 10⁻⁸ M or less, e.g. from 10⁻⁸ M to 10⁻¹³ M, e.g., from 10⁻⁹ M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C). In a non-adsorbent plate (Nunc #269620), 100 pM or 26 pM ¹²⁵I-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro l/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance assay. For example, an assay using a BIACORE (registered trademark)-2000 or a BIACORE (registered trademark)-3000 (BIAcore, Inc., Piscataway, NJ) is performed at 25 degrees C with immobilized antigen CM5 chips at ~10 response units (RU). In one embodiment, carboxymethylated dextran biosensor chips (CM5, BIACORE, Inc.) are activated with N-ethyl-N′- (3-dimethylaminopropyl)-carbodiimide hydrochloride (EDC) and N-hydroxysuccinimide (NHS) according to the supplier’s instructions. Antigen is diluted with 10 mM sodium acetate, pH 4.8, to 5 micro g/ml (~0.2 micro M) before injection at a flow rate of 5 micro l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C at a flow rate of approximately 25 micro l/min. Association rates (kₒₙ) and dissociation rates (k_{off}) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation = 295 nm; emission = 340 nm, 16 nm band-pass) at 25 degrees C of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

### 2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### 3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat′l Acad. Sci. USA 86:10029-10033 (1989); US Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall’Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal’s chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB (registered trademark) technology; U.S. Pat. No. 7,041,870 describing K-M MOUSE (registered trademark) technology, and U.S. Pat. Application Publication No. US 2007/0061900, describing VELOCIMOUSE (registered trademark) technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O′Brien et al., ed., Human Press, Totowa, NJ, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: US Pat. No. 5,750,373, and US Pat. Publication Nos. 2005/0079574, 2005/0119455, 2005/0266000, 2007/0117126, 2007/0160598, 2007/0237764, 2007/0292936, and 2009/0002360. Patent publications describing calcium concentration-dependent and/or pH-dependent antibody phage libraries include, for example: PCT Patent Publication No. WO 2013/046722.

Antibodies or antibody fragments isolated from human antibody libraries are considered human antibodies or human antibody fragments herein.

### 6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, bispecific antibodies may bind to two different epitopes of C5. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., US Pat. No. 4,676,980, and Brennan et al., Science, 229: 81 (1985)); using leucine zippers to produce bispecific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991). Techniques for making bispecific antibodies include, but are not limited to, in vitro post-production process employed in which IgG1 half-molecules recombine with other IgG1 half-molecules to generate bispecific IgG1 antibodies (see, e.g. Labrijn et al., J Immunol., 187: 3238 (2011)).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to C5 as well as another, different antigen (see, US 2008/0069820, for example).

### 7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| | | |
|---|---|---|
| Original Residue | Exemplary Substitutions | Preferred Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln(Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala, Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O′Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about +/- 3 amino acids upstream or downstream of position 297, i.e., between positions 294 and 300, due to minor sequence variations in antibodies. Such fucosylation variants may have improved ADCC function. See, e.g., U.S. Pat. Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); U.S. Pat. Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc gamma R binding (hence likely lacking ADCC activity), but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat′l Acad. Sci. USA 83:7059-7063 (1986)) and Hellstrom, I et al., Proc. Nat′l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96 (registered trademark) non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat′l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M.S. et al., Blood 101:1045-1052 (2003); and Cragg, M.S. and M.J. Glennie, Blood 103:2738-2743 (2004)). FcRn binding and in vivo clearance/half-life determinations can also be performed using methods known in the art (see, e.g., Petkova, S.B. et al., Int′l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in US Patent No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Antibodies may be produced using recombinant methods and compositions, e.g., as described in U.S. Pat. No. 4,816,567. In one embodiment, isolated nucleic acid encoding an anti-C5 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-C5 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-C5 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, NJ), pp. 255-268 (2003).

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals (usually non-human mammals) are immunized against the antigen, immunogenic conjugates, or derivatives by combining, e.g., 100 micro g or 5 micro g of the protein or conjugate (for rabbits or mice, respectively) with 3 volumes of Freund’s complete adjuvant and injecting the solution intradermally at multiple sites. One month later the animals are boosted with ⅕ to ⅒ the original amount of peptide or conjugate in Freund’s complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Monoclonal antibodies are obtained from a population of substantially homogeneous antibodies, i.e., the individual antibodies comprising the population are identical except for possible naturally occurring mutations and/or post-translational modifications (e.g., isomerizations, amidations) that may be present in minor amounts. Thus, the modifier “monoclonal” indicates the character of the antibody as not being a mixture of discrete antibodies.

For example, the monoclonal antibodies may be made using the hybridoma method first described by Kohler et al (1975) Nature 256(5517): 495-497. In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro.

The immunizing agent will typically include the antigenic protein or a fusion variant thereof. Generally either peripheral blood lymphocytes (PBLs) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press (1986), pp. 59-103).

Immortalized cell lines are usually transformed mammalian cells, particularly myeloma cells of rodent, bovine and human origin. Usually, rat or mouse myeloma cell lines are employed. The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which are substances that prevent the growth of HGPRT-deficient cells.

Preferred immortalized myeloma cells are those that fuse efficiently, support stable high-level production of antibody by the selected antibody-producing cells, and are sensitive to a medium such as HAT medium. Among these, preferred are murine myeloma lines, such as those derived from MOPC-21 and MPC-11 mouse tumors available from the Salk Institute Cell Distribution Center, San Diego, California USA, and SP-2 cells (and derivatives thereof, e.g., X63-Ag8-653) available from the American Type Culture Collection, Manassas, Virginia USA. Human myeloma and mouse-human heteromyeloma cell lines also have been described for the production of human monoclonal antibodies (Kozbor et al (1984) J Immunol 133(6): 3001-3005; Brodeur et al, Monoclonal Antibody Production Techniques and Applications, Marcel Dekker, Inc., New York (1987), pp. 51-63).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA). Such techniques and assays are known in the art. For example, binding affinity may be determined by the Scatchard analysis of Munson and Rodbard (1980) Anal Biochem 107(1): 220-239.

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as tumors in a mammal.

The monoclonal antibodies secreted by the subclones are suitably separated from the culture medium, ascites fluid, or serum by conventional immunoglobulin purification procedures such as, for example, protein A-Sepharose, hydroxyapatite chromatography, gel electrophoresis, dialysis, or affinity chromatography.

Antibodies may be produced by immunizing an appropriate host animal against an antigen. In one embodiment, the antigen is a polypeptide comprising a full-length C5. In one embodiment, the antigen is a polypeptide comprising the beta chain (SEQ ID NO: 1) or alpha chain (SEQ ID NO: 10) of C5. In one embodiment, the antigen is a polypeptide comprising the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or MG3-MG6 (SEQ ID NO: 9) domain of the beta chain of C5, or the anaphylatoxin domain (SEQ ID NO: 11) or C5-C345C/NTR domain (SEQ ID NO: 12) of the alpha chain of C5. In one embodiment, the antigen is a polypeptide comprising the region corresponding to the amino acids at positions 33 to 124 of the beta chain of C5 or a fragment consisting of amino acids 1-999 of the alpha chain (SEQ ID NO: 10) of C5. Also included in the present invention are antibodies produced by immunizing an animal against the antigen. The antibodies may incorporate any of the features, singly or in combination, as described in “Exemplary Anti-C5 Antibodies” above.

### C. Assays

Anti-C5 antibodies provided herein may be identified, screened for, or characterized for their physical/chemical properties and/or biological activities by various assays known in the art.

### 1. Binding Assays and Other Assays

In one aspect, an antibody of the invention is tested for its antigen binding activity, e.g., by known methods such as ELISA, Western blot, BIAcore, etc.

In another aspect, competition assays may be used to identify an antibody that competes or does not compete for binding to C5 or an epitope of C5 with any anti-C5 antibody described herein. In certain embodiments, when such a competing antibody is present in excess, it blocks (e.g., reduces) the binding of a reference antibody to C5 by at least 15%, 20%, 25%, 30%, 35%, 40%, 45%, 50%, 55%, 60%, 65%, 70%, 75% or more. In some instances, binding is inhibited by at least 80%, 85%, 90%, 95%, or more. In certain embodiments, when such a not-competing antibody is present in excess, it blocks (e.g., reduces) the binding of a reference antibody to C5 by at most 1%, 2%, 3%, 4%, 5%, 6%, 7%, 8%, 9%, 10%, 11%, 12%, 13%, 14% or less. In certain embodiments, such a competing antibody binds to the same epitope (e.g., a linear or a conformational epitope) that is bound by an anti-C5 antibody described herein (e.g., an anti-C5 antibody described in Table 2). Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized C5 is incubated in a solution comprising a first labeled antibody that binds to C5 and a second unlabeled antibody that is being tested for its ability to compete with the first antibody for binding to C5. The second antibody may be present in a hybridoma supernatant. As a control, immobilized C5 is incubated in a solution comprising the first labeled antibody but not the second unlabeled antibody. After incubation under conditions permissive for binding of the first antibody to C5, excess unbound antibody is removed, and the amount of label associated with immobilized C5 is measured. If the amount of label associated with immobilized C5 is substantially reduced in the test sample relative to the control sample, then that indicates that the second antibody is competing with the first antibody for binding to C5. See Harlow and Lane (1988) Antibodies: A Laboratory Manual ch. 14 (Cold Spring Harbor Laboratory, Cold Spring Harbor, NY).

In certain embodiments, whether an anti-C5 antibody of the present invention binds to a certain epitope can be determined as follows: C5 point mutants in which an amino acid (except for alanine) on C5 is substituted with alanine are expressed in 293 cells, and binding of an anti-C5 antibody to the C5 mutants is tested via ELISA, Western blot or BIAcore; wherein a substantial reduction or elimination of binding of the anti-C5 antibody to the C5 mutant relative to its binding to wild type C5 indicates that the anti-C5 antibody binds to an epitope comprising that amino acid on C5.

In another embodiment, whether an anti-C5 antibody with pH-dependent binding characteristics binds to a certain epitope can be determined as follows: C5 point mutants in which a histidine residue on C5 is substituted with another amino acid (e.g., tyrosine) are expressed in 293 cells, and binding of an anti-C5 antibody to the C5 mutants is tested via ELISA, Western blot or BIAcore; wherein a substantial reduction of binding of the anti-C5 antibody to wild type C5 at acidic pH relative to its binding to the C5 mutant at acidic pH, indicates that the anti-C5 antibody binds to an epitope comprising that histidine residue on C5. In further embodiments, binding of the anti-C5 antibody to wild type C5 at neutral pH is not substantially reduced relative to its binding to the C5 mutant at neutral pH.

### 2. Activity Assays

In one aspect, assays are provided for identifying anti-C5 antibodies thereof having biological activity. Biological activity may include, e.g., inhibiting the activation of C5, preventing the cleavage of C5 to form C5a and C5b, blocking the access of C5 convertase to the cleavage site on C5, blocking hemolytic activity caused by the activation of C5, etc. Antibodies having such biological activity in vivo and/or in vitro are also provided.

In certain embodiments, an antibody of the invention is tested for such biological activity.

In certain embodiments, whether a test antibody inhibits the cleavage of C5 into C5a and C5b, is determined by methods described in, e.g., Isenman et al (1980) J Immunol 124(1): 326-331. In another embodiment, this is determined by methods for specific detection of cleaved C5a and/or C5b proteins, e.g., ELISAs or Western blots. Where a decreased amount of a cleavage product of C5 (i.e., C5a and/or C5b) is detected in the presence of (or following contact with) the test antibody, the test antibody is identified as an antibody that can inhibit the cleavage of C5. In certain embodiments, the concentration and/or physiologic activity of C5a can be measured by methods, e.g., chemotaxis assays, RIAs, or ELISAs (See, e.g., Ward and Zvaifler (1971) J Clin Invest 50(3): 606-616).

In certain embodiments, whether a test antibody blocks the access of C5 convertase to C5 is determined by methods for the detection of protein interactions between the C5 convertase and C5, e.g., ELISAs or BIAcore. Where the interactions are decreased in the presence of (or following contact with) the test antibody, the test antibody is identified as an antibody that can block the access of C5 convertase to C5.

In certain embodiments, C5 activity can be measured as a function of its cell-lysing ability in a subject’s body fluids. The cell-lysing ability, or a reduction thereof, of C5 can be measured by methods well known in the art, for example, a conventional hemolytic assay, such as the hemolysis assay described by Kabat and Mayer (eds), Experimental Immunochemistry, 2nd Edition, 135-240, Springfield, IL, CC Thomas (1961), pages 135-139, or a conventional variation of that assay, such as the chicken erythrocyte hemolysis method as described in, e.g., Hillmen et al (2004) N Engl J Med 350(6): 552-559. In certain embodiments, C5 activity, or inhibition thereof, is quantified using a CH50eq assay. The CH50eq assay is a method for measuring the total classical complement activity in serum. This test is a lytic assay, which uses antibody-sensitized erythrocytes as the activator of the classical complement pathway, and various dilutions of the test serum to determine the amount required to give 50% lysis (CH50). The percentage of hemolysis can be determined, for example, using a spectrophotometer. The CH50eq assay provides an indirect measure of terminal complement complex (TCC) formation, since the TCC themselves are directly responsible for the hemolysis measured. Inhibition of C5 activation can also be detected and/or measured using the methods set forth and exemplified in the working examples. Using assays of these or other suitable types, candidate antibodies capable of inhibiting the activation of C5 can be screened. In certain embodiments, inhibition of C5 activation includes at least a 5%, 10%, 15%, 20%, 25%, 30%, 35%, or 40% or greater decrease in the C5 activation in an assay as compared to the effect of a negative control under similar conditions. In some embodiments, it refers to inhibition of C5 activation by at least 45%, 50%, 55%, 60%, 65%, 70%, 75%, 80%, 85%, 90%, or 95% or greater.

### 3. Assays to Test an Ability of a Combination of the Invention to Form an Antigen-Antibody Immune Complex Comprising at Least Two or More Antibodies

In one aspect, a combination of two or more antibodies of the invention is tested for its ability to form an antigen-antibody immune complex comprising at least two or more antibodies when the combination is contacted with those antigens e.g. C5. A combination of the anti-C5 antibodies of the invention can be contacted with C5 under the condition which allows them to form an antigen-antibody immune complex comprising at least two or more antibodies using conventional methodology by those skilled in the art (The Protein Protocols Handbook (Walker et al. eds.) 3rd edition (2009) Humana Press).

In certain embodiments, methods for testing the formation of an antigen-antibody immune complex comprising at least two or more antibodies include techniques in analytical chemistry, including methods that make use of the property of such an immune complex to become larger molecules than an antibody alone or an antigen molecule alone, such as size exclusion (gel filtration) chromatography, ultracentrifugation analysis method, light-scattering method, electron microscopy, and/or mass spectrometry (see e.g. Ferrant et al., Molecular Immunology (2002), 39, 77-84; see e.g. Oda et al., Molecular Immunology (2009), 47, 357-364). For example, when size exclusion (gel filtration) chromatography is used, whether an antigen-antibody immune complex comprising at least two or more antibodies is formed is tested by observing whether there are molecular species that are larger than those in analyses of the antigen molecule alone or the antibody molecule alone.

Furthermore, when the antibody or antigen has an immunoglobulin constant region, examples include immunochemical methods including methods that use the property of the immune complex to bind more strongly to an Fc receptor or a complement component than the antibody alone or the antigen alone, such as ELISA, FACS, or SPR methods (for example, methods using Biacore) (see e.g. Shields et al., The Journal of Biological Chemistry (2001) 276 (9), 6591-6604; see e.g., Singh et al., Journal of Immunological Methods (1982) 50, 109-114; see e.g. Suzuki et al., Journal of Immunology (2010) 184 (4), 1968-1976; see e.g. Luo et al., mAbs (2009) 1 (5) 491-504). For example, when ELISA is performed by immobilizing an Fc receptor, formation of an immune complex is tested by observing whether the detected signal is increased as compared to when an antigen molecule alone or an antibody molecule alone is tested.

### D. Immunoconjugates

The invention also provides immunoconjugates comprising an anti-C5 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶ , Re¹⁸⁸ , Sm¹⁵³, Bi²¹² , P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### E. Methods and Compositions for Diagnostics and Detection

In certain embodiments, any of the anti-C5 antibodies provided herein is useful for detecting the presence of C5 in a biological sample. The term “detecting” as used herein encompasses quantitative or qualitative detection. In certain embodiments, a biological sample comprises a cell or tissue, such as serum, whole blood, plasma, biopsy sample, tissue sample, cell suspension, saliva, sputum, oral fluid, cerebrospinal fluid, amniotic fluid, ascites fluid, milk, colostrums, mammary gland secretion, lymph, urine, sweat, lacrimal fluid, gastric fluid, synovial fluid, peritoneal fluid, ocular lens fluid and mucus.

In one embodiment, an anti-C5 antibody for use in a method of diagnosis or detection is provided. In a further aspect, a method of detecting the presence of C5 in a biological sample is provided. In certain embodiments, the method comprises contacting the biological sample with an anti-C5 antibody as described herein under conditions permissive for binding of the anti-C5 antibody to C5, and detecting whether a complex is formed between the anti-C5 antibody and C5. Such method may be an in vitro or in vivo method. In one embodiment, an anti-C5 antibody is used to select subjects eligible for therapy with an anti-C5 antibody, e.g. where C5 is a biomarker for selection of patients.

Exemplary disorders that may be diagnosed using an antibody of the invention include rheumatoid arthritis (RA); systemic lupus erythematosus (SLE); lupus nephritis; ischemia reperfusion injury (IRI); asthma; paroxysmal nocturnal hemoglobinuria (PNH); hemolytic uremic syndrome (HUS) (e.g., atypical hemolytic uremic syndrome (aHUS)); dense deposit disease (DDD); neuromyelitis optica (NMO); multifocal motor neuropathy (MMN); multiple sclerosis (MS); systemic sclerosis; macular degeneration (e.g., age-related macular degeneration (AMD)); hemolysis, elevated liver enzymes, and low platelets (HELLP) syndrome; thrombotic thrombocytopenic purpura (TTP); spontaneous fetal loss; epidermolysis bullosa; recurrent fetal loss; pre-eclampsia; traumatic brain injury; myasthenia gravis; cold agglutinin disease; Sjoegren’s syndrome; dermatomyositis; bullous pemphigoid; phototoxic reactions; Shiga toxin E. coli-related hemolytic uremic syndrome; typical or infectious hemolytic uremic syndrome (tHUS); C3 Glomerulonephritis; Antineutrophil cytoplasmic antibody (ANCA)-associated vasculitis; humoral and vascular transplant rejection; acute antibody mediated rejection (AMR); graft dysfunction; myocardial infarction; an allogenic transplant; sepsis; coronary artery disease; hereditary angioedema; dermatomyositis; Graves’ disease; atherosclerosis; Alzheimer’s disease (AD); Huntington’s disease; Creutzfeld-Jacob disease; Parkinson’s disease; cancers; wounds; septic shock; spinal cord injury; uveitis; diabetic ocular diseases; retinopathy of prematurity; glomerulonephritis; membranous nephritis; immunoglobulin A nephropathy; adult respiratory distress syndrome (ARDS); chronic obstructive pulmonary disease (COPD); cystic fibrosis; hemolytic anemia; paroxysmal cold hemoglobinuria; anaphylactic shock; allergy; osteoporosis; osteoarthritis; Hashimoto’s thyroiditis; type I diabetes; psoriasis; pemphigus; autoimmune hemolytic anemia (AIHA); idiopathic thrombocytopenic purpura (ITP); Goodpasture syndrome; Degos disease; antiphospholipid syndrome (APS); catastrophic APS (CAPS); a cardiovascular disorder; myocarditis; a cerebrovascular disorder; a peripheral vascular disorder; a renovascular disorder; a mesenteric/enteric vascular disorder; vasculitis; Henoch-Schoenlein purpura nephritis; Takayasu’s disease; dilated cardiomyopathy; diabetic angiopathy; Kawasaki’s disease (arteritis); venous gas embolus (VGE), restenosis following stent placement; rotational atherectomy; membraneous nephropathy; Guillain-Barre syndrome (GBS); Fisher syndrome; antigen-induced arthritis; synovial inflammation; viral infections; bacterial infections; fungal infections; and injury resulting from myocardial infarction, cardiopulmonary bypass and hemodialysis.

In certain embodiments, labeled anti-C5 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### F. Pharmaceutical Formulations

Pharmaceutical formulations of an anti-C5 antibody as described herein are prepared by mixing such antibody having the desired degree of purity with one or more optional pharmaceutically acceptable carriers (Remington’s Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in U.S. Pat. Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington’s Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

### G. Therapeutic Methods and Compositions

Any of the combination of the anti-C5 antibodies provided herein may be used in therapeutic methods.

In one aspect, a combination of two or more anti-C5 antibodies for use as a medicament is provided. In further aspects, a combination of two or more anti-C5 antibodies for use in treating a complement-mediated disease or condition which involves excessive or uncontrolled activation of C5 is provided. In certain embodiments, a combination of two or more anti-C5 antibodies for use in a method of treatment is provided. In certain embodiments, the invention provides a combination of two or more anti-C5 antibodies for use in a method of treating an individual having a complement-mediated disease or condition which involves excessive or uncontrolled activation of C5, comprising administering to the individual an effective amount of the combination of two or more anti-C5 antibodies. In one such embodiment, the method further comprises administering to the individual an effective amount of at least one additional therapeutic agent.

When the antigen is a soluble protein, the binding of an antibody to its antigen can result in an extended half-life of the antigen in plasma (i.e., reduced clearance of the antigen from plasma), since the antibody itself has a longer half-life in plasma and serves as a carrier for the antigen. This is due to the recycling of the antigen-antibody complex by FcRn through the endosomal pathway in cell (Roopenian and Akilesh (2007) Nat Rev Immunol 7(9): 715-725). However, an antibody with pH-dependent binding characteristics, which binds to its antigen in neutral extracellular environment while releasing it into acidic endosomal compartments following entry into cells, is expected to have superior properties in terms of antigen neutralization and clearance relative to its counterpart that binds in a pH-independent manner (Igawa et al (2010) Nature Biotechnol 28(11); 1203-1207; Devanaboyina et al (2013) mAbs 5(6): 851-859; International Patent Application Publication No: WO 2009/125825).

In further embodiments, the invention provides a combination of two or more anti-C5 antibodies for use in enhancing the clearance of C5 from plasma. In certain embodiments, the invention provides a combination of two or more anti-C5 antibodies for use in a method of enhancing the clearance of C5 from plasma in an individual comprising administering to the individual an effective amount of the combination of two or more anti-C5 antibodies to enhance the clearance of C5 from plasma. In one embodiment, a combination of two or more anti-C5 antibodies enhances the clearance of C5 from plasma, compared to a conventional anti-C5 antibody which does not have pH-dependent binding characteristics. An “individual” according to any of the above embodiments is preferably a human.

In further embodiments, the invention provides a combination of two or more anti-C5 antibodies for use in suppressing the accumulation of C5 in plasma. In certain embodiments, the invention provides a combination of two or more anti-C5 antibodies for use in a method of suppressing the accumulation of C5 in plasma in an individual, comprising administering to the individual an effective amount of the combination of two or more anti-C5 antibodies to suppress the accumulation of C5 in plasma. In one embodiment, the accumulation of C5 in plasma is the result of the formation of an antigen-antibody complex. In another embodiment, a combination of two or more anti-C5 antibodies suppresses the accumulation of C5 in plasma, compared to a conventional anti-C5 antibody which does not have pH-dependent binding characteristics. An “individual” according to any of the above embodiments is preferably a human.

A combination of two or more anti-C5 antibodies of the present invention may inhibit the activation of C5. In further embodiments, the invention provides a combination of two or more anti-C5 antibodies for use in inhibiting the activation of C5. In certain embodiments, the invention provides a combination of two or more anti-C5 antibodies for use in a method of inhibiting the activation of C5 in an individual, comprising administering to the individual an effective amount of the combination of two or more anti-C5 antibodies to inhibit the activation of C5. In one embodiment, the cytotoxicity mediated by C5 is suppressed by inhibiting the activation of C5. An “individual” according to any of the above embodiments is preferably a human.

In a further aspect, the invention provides for the use of a combination of two or more anti-C5 antibodies in the manufacture or preparation of a medicament. In one embodiment, the medicament is for treatment of a complement-mediated disease or condition which involves excessive or uncontrolled activation of C5. In a further embodiment, the medicament is for use in a method of treating a complement-mediated disease or condition which involves excessive or uncontrolled activation of C5, comprising administering to an individual having a complement-mediated disease or condition which involves excessive or uncontrolled activation of C5 an effective amount of the medicament. In one such embodiment, the method further comprises administering to the individual an effective amount of at least one additional therapeutic agent. An “individual” according to any of the above embodiments is preferably a human.

In a further embodiment, the medicament is for enhancing the clearance of C5 from plasma. In a further embodiment, the medicament is for use in a method of enhancing the clearance of C5 from plasma in an individual comprising administering to the individual an effective amount of the medicament to enhance the clearance of C5 from plasma. In one embodiment, a combination of two or more anti-C5 antibodies enhances the clearance of C5 from plasma, compared to a conventional anti-C5 antibody which does not have pH-dependent binding characteristics. An “individual” according to any of the above embodiments may be a human.

In a further embodiment, the medicament is for suppressing the accumulation of C5 in plasma. In a further embodiment, the medicament is for use in a method of suppressing the accumulation of C5 in plasma in an individual, comprising administering to the individual an effective amount of the medicament to suppress the accumulation of C5 in plasma. In one embodiment, the accumulation of C5 in plasma is a result of the formation of an antigen-antibody complex. In another embodiment, a combination of two or more anti-C5 antibodies suppresses the accumulation of C5 in plasma, compared to a conventional anti-C5 antibody which does not have pH-dependent binding characteristics. An “individual” according to any of the above embodiments may be a human.

A combination of two or more anti-C5 antibodies of the present invention may inhibit the activation of C5. In a further embodiment, the medicament is for inhibiting the activation of C5. In a further embodiment, the medicament is for use in a method of inhibiting the activation of C5 in an individual, comprising administering to the individual an effective amount of the medicament to inhibit the activation of C5. In one embodiment, the cytotoxicity mediated by C5 is suppressed by inhibiting the activation of C5. An “individual” according to any of the above embodiments may be a human.

In a further aspect, the invention provides a method for treating a complement-mediated disease or condition which involves excessive or uncontrolled activation of C5. In one embodiment, the method comprises administering to an individual having such a complement-mediated disease or condition which involves excessive or uncontrolled activation of C5 an effective amount of a combination of two or more anti-C5 antibodies. In one such embodiment, the method further comprises administering to the individual an effective amount of at least one additional therapeutic agent. An “individual” according to any of the above embodiments may be a human.

In a further aspect, the invention provides a method for enhancing the clearance of C5 from plasma in an individual. In one embodiment, the method comprises administering to the individual an effective amount of a combination of two or more anti-C5 antibodies to enhance the clearance of C5 from plasma. In one embodiment, a combination of two or more anti-C5 antibodies enhances the clearance of C5 from plasma, compared to a conventional anti-C5 antibody which does not have pH-dependent binding characteristics. In one embodiment, an “individual” is a human.

In a further aspect, the invention provides a method for suppressing the accumulation of C5 in plasma in an individual. In one embodiment, the method comprises administering to the individual an effective amount of a combination of two or more anti-C5 antibodies to suppress the accumulation of C5 in plasma. In one embodiment, the accumulation of C5 in plasma is a result of the formation of an antigen-antibody complex. In another embodiment, a combination of two or more anti-C5 antibodies suppresses the accumulation of C5 in plasma, compared to a conventional anti-C5 antibody which does not have pH-dependent binding characteristics. In one embodiment, an “individual” is a human.

A combination of two or more anti-C5 antibodies of the present invention may inhibit the activation of C5. In a further aspect, the invention provides a method for inhibiting the activation of C5 in an individual. In one embodiment, the method comprises administering to the individual an effective amount of a combination of two or more anti-C5 antibodies to inhibit the activation of C5. In one embodiment, the cytotoxicity mediated by C5 is suppressed by inhibiting the activation of C5. In one embodiment, an “individual” is a human.

Two or more anti-C5 antibodies comprised in the combination of the invention may be formulated in one composition or in separate compositions. Two or more anti-C5 antibodies comprised in the combination of the invention formulated in separate compositions may be administered into the individual at the same or different time point. Administration of two or more anti-C5 antibodies comprised in the combination of the invention typically is carried out over a defined time period (usually minutes, hours, days or weeks depending upon the combination selected). The combination of the invention is intended to embrace administration of two or more anti-C5 antibodies comprised in a sequential manner, that is, administration of each anti-C5 antibody at a different time (in any order), as well as administration of two or moreanti-C5 antibodies in a concurrent (simultaneous) manner. Concurrent administration may be as separate pharmaceutical formulations or as a single dosage form (e.g., as a single pharmaceutical formulation. In some embodiments, other one or more anti-C5 antibodies are administered once a day, for example, in the morning or in the evening. In some embodiments, other one or more anti-C5 antibodies are administered once a day at any time of day. In some embodiments, the second 960 mg dose (e.g., four 240 mg containers) of anti-C5 antibody I is about 12 hours after the first 960 mg dose (e.g., four 240 mg containers) of anti-C5 antibody I. In some embodiments, anti-C5 antibody I is administered once in the morning and once in the evening.

In a further aspect, the invention provides pharmaceutical formulations comprising any of two or more anti-C5 antibodies comprised in the combination provided herein, e.g., for use in any of the above therapeutic methods. In one embodiment, a pharmaceutical formulation comprises any of two or more anti-C5 antibodies comprised in the combination provided herein and a pharmaceutically acceptable carrier. In another embodiment, a pharmaceutical formulation comprises any of two or more anti-C5 antibodies comprised in the combination provided herein and at least one additional therapeutic agent. In a further aspect, the invention provides pharmaceutical formulations comprising the combination of two or more anti-C5 antibodies provided herein, e.g., for use in any of the above therapeutic methods. In one embodiment, a pharmaceutical formulation comprises the combination of two or more anti-C5 antibodies provided herein and a pharmaceutically acceptable carrier. In another embodiment, a pharmaceutical formulation comprises the combination of two or more anti-C5 antibodies provided herein and at least one additional therapeutic agent.

In certain embodiments, a complement-mediated disease or condition which involves excessive or uncontrolled activation of C5 is selected from the group consisting of rheumatoid arthritis (RA); systemic lupus erythematosus (SLE); lupus nephritis; ischemia reperfusion injury (IRI); asthma; paroxysmal nocturnal hemoglobinuria (PNH); hemolytic uremic syndrome (HUS) (e.g., atypical hemolytic uremic syndrome (aHUS)); dense deposit disease (DDD); neuromyelitis optica (NMO); multifocal motor neuropathy (MMN); multiple sclerosis (MS); systemic sclerosis; macular degeneration (e.g., age-related macular degeneration (AMD)); hemolysis, elevated liver enzymes, and low platelets (HELLP) syndrome; thrombotic thrombocytopenic purpura (TTP); spontaneous fetal loss; epidermolysis bullosa; recurrent fetal loss; pre-eclampsia; traumatic brain injury; myasthenia gravis; cold agglutinin disease; Sjoegren’s syndrome; dermatomyositis; bullous pemphigoid; phototoxic reactions; Shiga toxin E. coli-related hemolytic uremic syndrome; typical or infectious hemolytic uremic syndrome (tHUS); C3 Glomerulonephritis; Antineutrophil cytoplasmic antibody (ANCA)-associated vasculitis; humoral and vascular transplant rejection; acute antibody mediated rejection (AMR); graft dysfunction; myocardial infarction; an allogeneic transplant; sepsis; coronary artery disease; hereditary angioedema; dermatomyositis; Graves’ disease; atherosclerosis; Alzheimer’s disease (AD); Huntington’s disease; Creutzfeld-Jacob disease; Parkinson’s disease; cancers; wounds; septic shock; spinal cord injury; uveitis; diabetic ocular diseases; retinopathy of prematurity; glomerulonephritis; membranous nephritis; immunoglobulin A nephropathy; adult respiratory distress syndrome (ARDS); chronic obstructive pulmonary disease (COPD); cystic fibrosis; hemolytic anemia; paroxysmal cold hemoglobinuria; anaphylactic shock; allergy; osteoporosis; osteoarthritis; Hashimoto’s thyroiditis; type I diabetes; psoriasis; pemphigus; autoimmune hemolytic anemia (AIHA); idiopathic thrombocytopenic purpura (ITP); Goodpasture syndrome; Degos disease; antiphospholipid syndrome (APS); catastrophic APS (CAPS); a cardiovascular disorder; myocarditis; a cerebrovascular disorder; a peripheral vascular disorder; a renovascular disorder; a mesenteric/enteric vascular disorder; vasculitis; Henoch-Schoenlein purpura nephritis; Takayasu’s disease; dilated cardiomyopathy; diabetic angiopathy; Kawasaki’s disease (arteritis); venous gas embolus (VGE), restenosis following stent placement; rotational atherectomy; membranous nephropathy; Guillain-Barre syndrome (GBS); Fisher syndrome; antigen-induced arthritis; synovial inflammation; viral infections; bacterial infections; fungal infections; and injury resulting from myocardial infarction, cardiopulmonary bypass and hemodialysis.

A combination of two or more antibodies of the invention can be used either alone or in combination with other agents in a therapy. For instance, a combination of two or more antibodies of the invention may be co-administered with at least one additional therapeutic agent.

Such combination therapies noted above encompass combined administration (where two or more therapeutic agents are included in the same or separate formulations), and separate administration, in which case, administration of the combination of two or more antibodies of the invention can occur prior to, simultaneously, and/or following, administration of the additional therapeutic agent or agents. In one embodiment, administration of the combination of two or more anti-C5 antibodies and administration of an additional therapeutic agent occur within about one month, or within about one, two or three weeks, or within about one, two, three, four, five, or six days, of each other.

A combination of two or more antibodies of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

A combination of two or more antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The combination of two or more antibodies need not be, but is optionally formulated with one or more agents currently used to prevent or treat the disorder in question. The effective amount of such other agents depends on the amount of each antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a combination of two or more antibodies of the invention (when used alone or in combination with one or more other additional therapeutic agents) will depend on the type of disease to be treated, the type of the combination of two or more antibodies, the severity and course of the disease, whether the combination of two or more antibodies is administered for preventive or therapeutic purposes, previous therapy, the patient’s clinical history and response to the combination of two or more antibodies, and the discretion of the attending physician. The combination of two or more antibodies is suitably administered to the patient at one time or over a series of treatments. Depending on the type and severity of the disease, about 1 micro g/kg to 15 mg/kg (e.g. 0.1 mg/kg-10 mg/kg) of each antibody can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the combination of two or more antibodies would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the combination of two or more antibodies). An initial higher loading dose, followed by one or more lower doses may be administered. The progress of this therapy is easily monitored by conventional techniques and assays.

It is understood that any of the above formulations or therapeutic methods may be carried out using an immunoconjugate of the invention in place of or in addition to each anti-C5 antibody comprised in the combination of the invention.

### H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody comprised in the combination of the invention. The label or package insert indicates that the composition is used for treating the condition of choice. The label or package insert may also indicate that the composition is used for treating the condition of choice as a combination with the other active agent in the composition which is the other antibody comprised in the combination of the invention. The article of manufacture may comprise (a) a first container with a composition contained therein, wherein the composition comprises an antibody comprised in the combination of the invention; and (b) a second container with a composition contained therein, wherein the composition comprises another antibody comprised in the combination of the invention. The article of manufacture may comprise a first, a second and a third container with a composition contained therein, wherein the composition comprises a first, a second and a third antibody comprised in the combination of the invention, respectively. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer’s solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to a combination of two or more anti-C5 antibodies.

### EXAMPLES

The following are examples of methods and compositions of the invention. It is understood that various other embodiments may be practiced, given the general description provided above.

### Example 1

### Preparation of C5 Expression and Purification of Recombinant Human and Cynomolgus Monkey C5

Recombinant human C5 (NCBI GenBank accession number: NP_001726.2, SEQ ID NO: 13) was expressed transiently using FreeStyle293-F cell line (Thermo Fisher, Carlsbad, CA, USA). Conditioned media expressing human C5 was diluted with equal volume of milliQ water, then applied to a Q-sepharose FF or Q-sepharose HP anion exchange column (GE healthcare, Uppsala, Sweden), followed by elution with NaCl gradient. Fractions containing human C5 were pooled, then salt concentration and pH was adjusted to 80 mM NaCl and pH6.4, respectively. The resulting sample was applied to a SP-sepharose HP cation exchange column (GE healthcare, Uppsala, Sweden) and eluted with a NaCl gradient. Fractions containing human C5 were pooled and subjected to CHT ceramic Hydroxyapatite column (Bio-Rad Laboratories, Hercules, CA, USA). Human C5 eluate was then applied to a Superdex 200 gel filtration column (GE healthcare, Uppsala, Sweden). Fractions containing human C5 was pooled and stored at -150° C. Either in-house prepared recombinant human C5 or plasma derived human C5 (CALBIOCHEM, Cat#204888) was used for the study.

Expression and purification of recombinant cynomolgus monkey C5 (NCBI GenBank accession number: XP_005580972, SEQ ID NO: 14) was done exactly the same way as the human counterpart.

### Example 2

### Preparation of Synthetic Calcium Library

A gene library of antibody heavy chain variable regions which were used as synthetic human heavy chain libraries consist of 10 heavy chain libraries. Germ-line frameworks VH1-2, VH1-69, VH3-23, VH3-66, VH3-72, VH4-59, VH4-61, VH4-b, VH5-51, and VH6-1 were selected for this library based on germ-line frequency in human B-cell repertoires, and biophysical properties of V-gene families. The synthetic human heavy chain library was diversified at the antibody-binding site mimicking human B cell antibody repertoires.

A gene library of antibody light chain variable regions were designed to have calcium binding motif and were diversified at the positions which would contribute to antigen recognition, referring to human B cell antibody repertoires. The design of a gene library of antibody light chain variable regions which exert characteristics for calcium-dependent binding to antigens is described in WO 2012/073992.

The combination of a heavy chain variable region library and a light chain variable region library is inserted in a phagemid vector, and a phage library was constructed, referring to (Methods Mol Biol. (2002) 178, 87-100). A trypsin-cleavage site was introduced into the phagemid vector at a linker region between Fab and pIII protein. Modified M13KO7 helper phage which has a trypsin-cleavage site between N2 and CT domains at geneIII was used for Fab displayed phage preparation.

### Example 3

### Isolation of Calcium Dependent Anti-C5 Antibodies

The phage display library was diluted with TBS supplemented with BSA and CaCl₂ at the final concentration of 4% and 1.2 mM, respectively. As a panning method, conventional magnetic beads selection was applied referring to general protocols (J. Immunol. Methods. (2008) 332 (1-2), 2-9, J. Immunol. Methods. (2001) 247 (1-2), 191-203, Biotechnol. Prog. (2002) 18(2) 212-20, Mol. Cell Proteomics (2003) 2 (2), 61-9). As magnetic beads, NeutrAvidin coated beads (Sera-Mag SpeedBeads NeutrAvidin-coated) or Streptavidin coated beads (Dynabeads M-280 Streptavidin) were applied. Human C5 (CALBIOCHEM, Cat#204888) was labelled with EZ-Link NHS-PEG4-Biotin (PIERCE, Cat No. 21329).

The initial round of phage selection, the phage display library was incubated with biotinylated human C5 (312.5 nM) for 60 minutes at room temperature. Phages that displayed binding Fab variants were then captured using magnetic beads.

After incubation with beads for 15 minutes at room temperature, the beads were washed three times with 1 mL of TBS containing 1.2 mM CaCl₂ and 0.1% Tween20, and the beads were washed twice with 1 mL of TBS containing 1.2 mM CaCl₂. Phages were eluted by re-suspending the beads with TBS containing 1 mg/mL trypsin for 15 minutes. The eluted phages were infected with ER2738 and rescued by the helper phage. The rescued phages were precipitated with polyethylene glycol, re-suspended with TBS supplemented with BSA and CaCl₂ at the final concentration of 4% and 1.2 mM, respectively and used in the next round of panning.

After 1st round of panning, the phages were selected for its calcium dependency, in which the antibody binds to C5 stronger in the presence of calcium ion. In the second and third round, the panning was performed in the same manner as the first round except by using 50 nM (second round) or 12.5 nM (third round) of biotinylated antigen and finally eluted with 0.1 mL of elution buffer (50 mM MES, 2 mM EDTA, 150 mM NaCl, pH5.5) and contacted with 1 micro L of 100 mg/mL trypsin to select for its calcium dependency. After selection, selected phage clones were converted to IgG format.

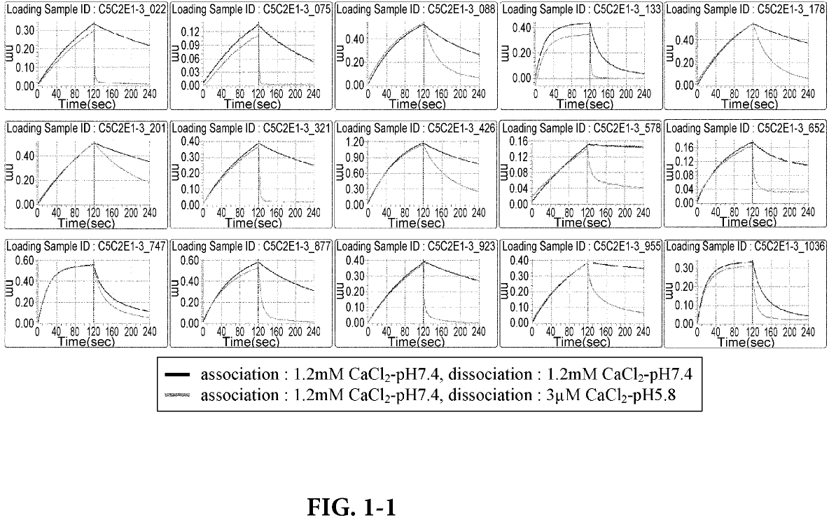

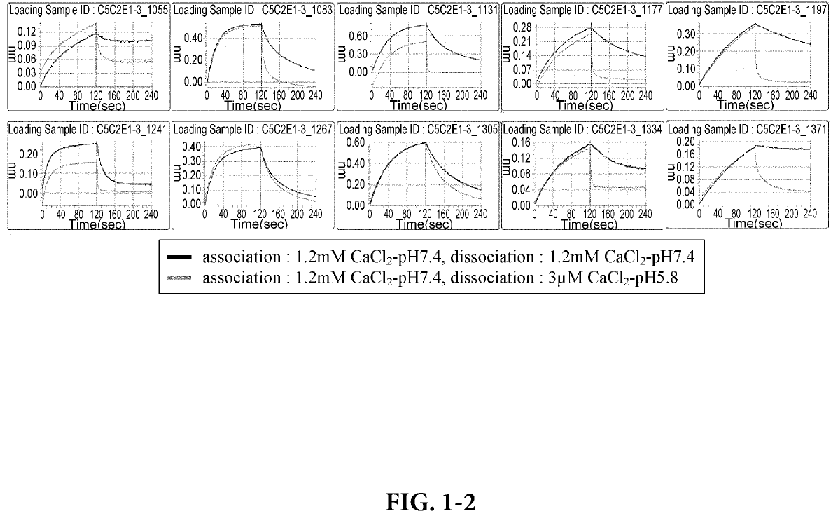

Binding ability of converted IgG antibodies against human C5 were assessed under two different conditions: association and dissociation at 1.2 mM CaCl₂-pH 7.4 (20 mM MES, 150 mM NaCl, 1.2 mM CaCl₂) and association at 1.2 mM CaCl₂-pH 7.4 (20 mM MES, 150 mM NaCl, 1.2 mM CaCl₂) and dissociation at 3 micro M CaCl₂-pH 5.8 (20 mM MES, 150 mM NaCl, 3 micro M CaCl₂), at 30° C. using Octet RED384 system (Pall Life Sciences). 25 clones of pH-Calcium dependent antigen binding clones were isolated. The sensorgrams of these antibodies are shown in FIG. 1.

### Example 4

### Identification of Anti-C5 Bispecific Antibody Which Can Form Multimeric Antigen-Antibody Immune Complex (Ag-Ab IC)

### 4.1. Preparation of Antibody Expression Vector and Expression and Purification of Recombinant Antibodies

From the clones isolated in Example 3, nine pH or calcium dependent anti-C5 antibody clones were selected for further analysis (CFP0008, 0011, 0015, 0016, 0017, 0018, 0019, 0020, 0021). Some amino acid substitutions were introduced to CFP0016 heavy chain variable region by a method generally known to those skilled in the art to improve properties of the antibodies like physicochemical properties. This CFP0016 variant, CFP0016H019, was used for further analysis instead of CFP0016. The amino acid sequences of VH and VL regions of these nine antibodies are described in Table 2. In this table, names described in brackets represent the abbreviated names.

TABLE 2

Clone name and amino acid sequence of selected antibodies

| | | | | |
|---|---|---|---|---|
| Clone name | VH name | VH SEQ ID | VL name | VL SEQ ID |
| CFP0008 (08) | CFP0008H (08H) | NO: 15 | CFP0008L (08L) | NO: 16 |
| CFP0011 (11) | CFP0011H (11H) | NO: 17 | CFP0011L (11L) | NO: 18 |
| CFP0015 (15) | CFP0015H (15H) | NO: 19 | CFP0015L (15L) | NO: 20 |
| CFP0016H019 (16H019) | CFP0016H019 (16H019) | NO: 21 | CFP0016L (16L) | NO: 22 |
| CFP0017 (17) | CFP0017H (17H) | NO: 23 | CFP0017L (17L) | NO: 24 |
| CFP0018 (18) | CFP0018H (18H) | NO: 25 | CFP0018L (18L) | NO: 26 |
| CFP0019 (19) | CFP0019H (19H) | NO: 27 | CFP0019L (19L) | NO: 28 |
| CFP0020 (20) | CFP0020H (20H) | NO: 29 | CFP0020L (20L) | NO: 30 |
| CFP0021 (21) | CFP0021H (21H) | NO: 31 | CFP0021L (21L) | NO: 32 |

The full-length genes having nucleotide sequences encoding antibody heavy chain and light chain were synthesized and prepared by a method generally known to those skilled in the art. Heavy chain and light chain expression vectors were prepared by inserting the obtained plasmid fragments into vectors for expression in mammalian cells. The obtained expression vectors were sequenced by a method generally known to those skilled in the art. For expression of antibodies, the prepared plasmids were transiently transfected to FreeStyle293-F cell line (Thermo Fisher Scientific). Purification from the conditioned media expressing antibodies was conducted by a method generally known to those skilled in the art using rProtein A Sepharose Fast Flow (GE Healthcare).

### 4.2. Generation of Anti-C5 Bispecific Antibody

Bispecific antibodies, which potentially recognize two different epitopes of C5, were generated by combination of two different clones described in Table 2. Bispecific antibodies were prepared as IgG format having two different clones of Fab in each binding site of the antibody. In these bispecific IgG antibodies, two heavy chains comprise distinct heavy chain constant regions (F760G4P1, SEQ ID NO: 33 and F760G4N1, SEQ ID NO: 34) from each other so as to efficiently form a heterodimer of the two heavy chains. Potential bispecific antibodies, which are twenty-one bispecific antibodies constructed by combinations of two binding sites comprising the heavy chain and the light chain of nine monoclonal antibodies (MAbs) described in Table 2, were prepared using a method generally known to those skilled in the art. The anti-C5 bispecific antibody comprising the binding sites of anti-C5 MAb “X” and anti-C5 MAb “Y” is represented as “X//Y”.

### 4.3. Evaluation of Avidity Effect by Multimeric Ag-Ab IC Formation

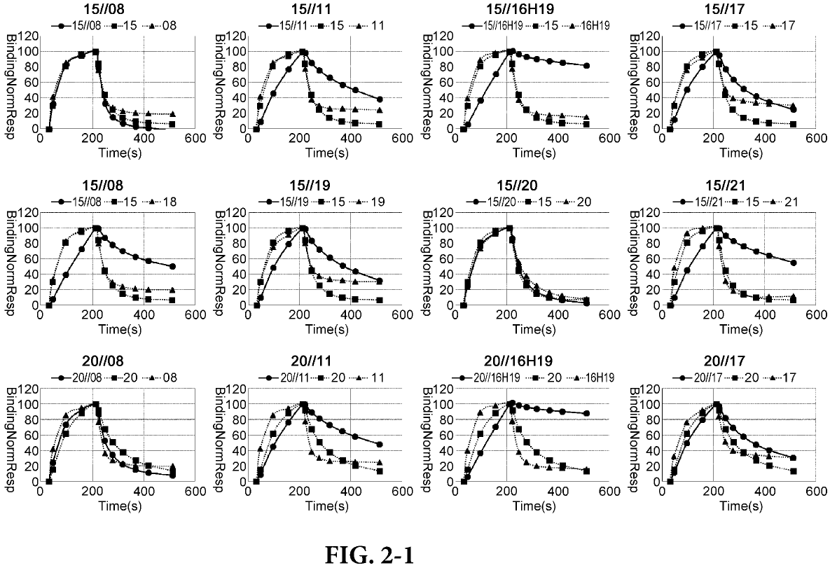

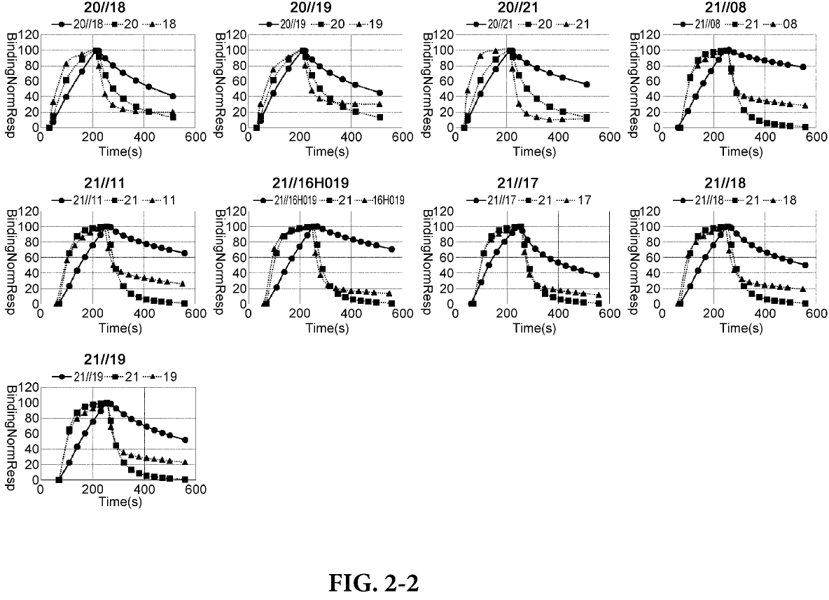

Ag-Ab IC containing more than two antibodies or Fcs can bind to Fc receptors (FcRn or Fc gamma receptor) by multivalent avidity binding. Here, we referred Ag-Ab IC comprising more than two antibodies or Fcs as multimeric or large Ag-Ab IC. To evaluate the avidity effect by formation of multimeric Ag-Ab IC, mouse FcRn (recombinant produced by a method generally known to those skilled in the art, and hereinafter, referred to as mFcRn) was immobilized onto Series S Sensor Chip CM5 (GE Healthcare, Cat No. BR-1005-30) by the amine coupling method. Anti-C5 MAbs or bispecific antibodies prepared above were contacted with human C5 in approximately one to one ratio in molar concentration, and incubated for about 30 minutes at room temperature to reach equilibrium of Ag-Ab IC formation. The binding of the Ag-Ab IC against immobilized mFcRn at pH 7.4 and at 37° C. were assessed using Biacore T200 instrument (GE Healthcare) or Biacore 4000 instrument (GE Healthcare). The running buffer used was pH 7.4 ACES Buffer containing 1.2 mM Ca (20 mM ACES, 150 mM NaCl, 1.2 mM CaCl₂, 0.05% Tween 20). In order to compare the dissociation rate of Ag-Ab IC from immobilized mFcRn, binding normalized response was used, which is determined by subtracting baseline response (the value determined by this step is referred to as baseline normalized response), and then normalizing the baseline normalized response with the value at the last time point of association phase as 100. The obtained binding normalized responses comparing anti-C5 bispecific antibodies and two anti-C5 MAbs which give origin for binding sites of the bispecific antibody are shown in FIG. 2.

All of the anti-C5 MAbs tested showed rapid dissociation from mFcRn due to weak monomeric interaction or affinity binding of Ag-Ab IC of Mab to mFcRn. On the other hand, most of the anti-C5 bispecific antibodies tested showed slower dissociation than anti-C5 MAbs due to multimeric interaction or avidity binding of Ag-Ab IC of bispecific antibody to mFcRn. This result suggested that these anti-C5 bispecific antibodies which showed slower dissociation formed multimeric Ag-Ab IC by recognizing two different epitopes on the same C5 molecule.. On the other hand, some bispecific combinations (15//08, 15//20 and 20//08) showed rapid dissociation from mFcRn similar to MAbs which give origin for binding sites of the bispecific antibody (15//08, 15//20 and 20//08), thus these bispecific antibodies could not form multimeric Ag-Ab IC.

### Example 5

### Light Chain Commonization

### 5.1. Generation and Evaluation of Light Chain Variants

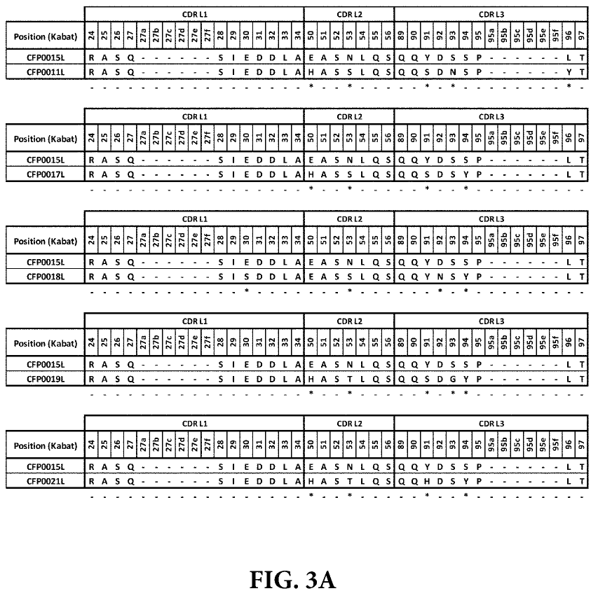

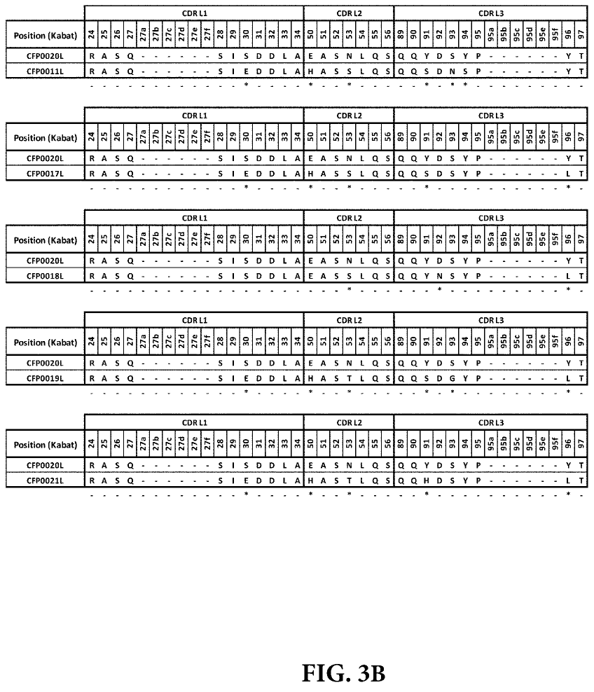

Anti-C5 bispecific antibodies appropriate for accelerating the clearance of C5 found in Example 4 comprised two binding sites whose two heavy chains and two light chains were distinct from each other. In this embodiment, anti-C5 bispecific antibodies whose binding sites comprise common light chain e.g. light chain whose sequence of two binding sites is identical are provided (PLoS One. 2013;8(2):e57479). Ten clones of anti-C5 bispecific antibodies (15//11, 15//17, 15//18, 15//19, 15//21, 20//11, 20//17, 20//18, 20//19 and 20//21) were selected for light chain commonization. To identify the common light chain for these anti-C5 bispecific antibodies, a number of light chain variants were generated by introducing amino acid substitution(s) into light chain CDR by a method generally known to those skilled in the art. The amino acid substitutions were mainly introduced at the positions where amino acid residues are different between sequences of two light chains which give origin for binding sites of the bispecific antibody. Comparisons of the CDR sequence between the two light chains are shown in FIG. 3. In this figure, * indicates the residues which are different between the two light chains. The light chain variants were tested for the binding affinity to C5 at pH 7.4 and at 37° C. using Biacore T200 instrument (GE Healthcare) or Biacore 4000 instrument (GE Healthcare). Protein A/G (Pierce, Cat No. #21186) or anti-human IgG (Fc) antibody (within Human Antibody Capture Kit; GE Healthcare, Cat No. BR-1008-39) was immobilized onto a Series S CM4 (GE Healthcare, Cat No. BR-1005-34) by amine coupling method. Anti-C5 antibodies were captured on an immobilized molecule, and then human C5 was injected. The running buffer used was pH 7.4 ACES Buffer containing 1.2 mM Ca (20 mM ACES, 150 mM NaCl, 1.2 mM CaCl₂, 0.05% Tween 20). The obtained results are shown in Table 3. A value, %binding, was determined by normalizing binding response with that of antibody comprising parent light chain as 100. From this substitution study, replacement to the same amino acid at the same position which were acceptable for both light chains could be identified.

### 5.2. Identification of Common Light Chain for 20//18

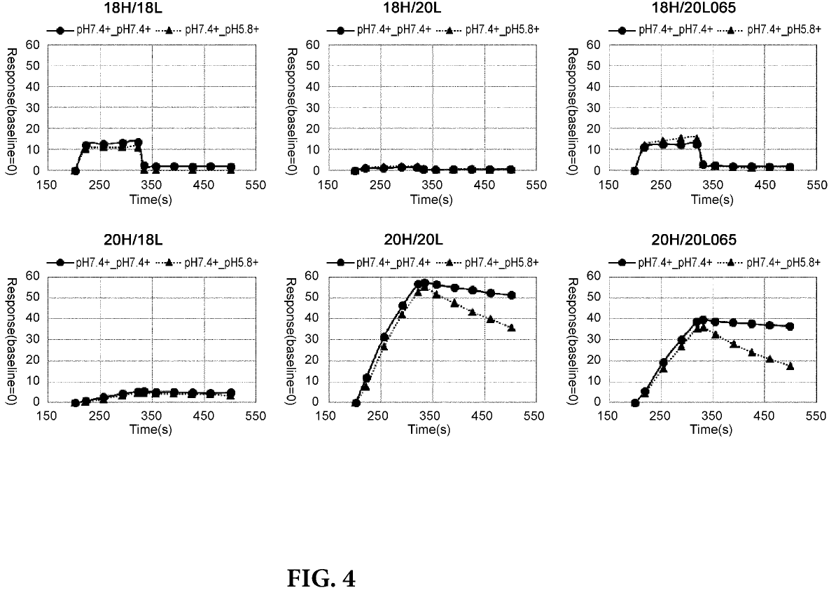

In comparison of the sequence of two light chains of 20//18 bispecific antibody, three amino acid residues at positions 53, 92 and 96 (designated according to Kabat numbering) were different, these residues were necessary to be commonized. From the analysis of binding activity to C5 of anti-C5 Mab light chain variants, His, Asn, Ser or Thr at position 53, Asp, Asn or Ser at position 92 and/or Phe, His, Trp or Tyr at position 96 were identified as acceptable residues for common light chain which maintains C5 binding affinity. A light chain having combination of these acceptable residues at positions 53, 92 and 96, 20L065 (SEQ ID NO: 35), was identified as one of common light chains for 20//18. Then, two antibodies comprising heavy chain of clone 20 and light chain of 20L065, and heavy chain of clone 18 and light chain of 20L065 were prepared as previously described. Binding sensorgrams of two antibodies comprising common light chain e.g. 20L065 were shown in FIG. 4 comparing to binding sensorgrams of antibodies comprising parent light chain. The common light chain 20L065 maintains C5 binding affinity for heavy chains of both clone 20 and 18.

TABLE 3

Binding analysis of light chain variants (%binding, parent antibody as 100) Positions of residues are designated according to Kabat numbering.

| Position | Mutation | 15L | 20L | 11L | 17L | 18L | 19L | 21L |
|---|---|---|---|---|---|---|---|---|
| - | - | 100 | 100 | 100 | 100 | 100 | 100 | 100 |
| 30 | 30A | 25.7 | 97.4 | 100.1 | 79.9 | 21.2 | 7.3 | 76.9 |
| | 30D | 59.9 | 73.7 | 71.4 | 89.4 | 17.0 | -14.8 | 87.5 |
| | 30E | 100 | 63.6 | 100 | 100 | 20.8 | 100 | 100 |
| | 30F | 30.7 | - | - | - | 85.2 | - | - |
| | 30H | 18.4 | 105.3 | 13.6 | 52.1 | 87.2 | -0.2 | 59.7 |
| | 30N | 21.4 | 104.2 | - | - | 200.8 | - | - |
| | 30P | 609 | 57.1 | - | - | 111.2 | - | - |
| | 30Q | 42.6 | 96.1 | - | - | 129.9 | - | - |
| | 30S | 23.1 | 100 | 44.5 | 79.1 | 100 | 3.6 | 76.7 |
| | 30T | 25.1 | - | - | - | 90.7 | - | - |
| | 30Y | 51.3 | - | - | - | 82.4 | - | - |
| 31 | 31D | 100 | 100 | 100 | 100 | 100 | 100 | 100 |
| | 31A | 30.0 | 12.1 | 112.1 | 0.6 | 0.7 | 7.1 | 31.2 |
| | 31E | 22.4 | 25.9 | - | - | - | - | - |
| | 31H | 59.4 | 6.7 | 100.3 | 3.4 | 9.2 | 7.6 | 41.3 |
| | 31N | 42.2 | 114.3 | 124.8 | - | 129.9 | - | 52.3 |
| | 31Q | 25.8 | 34.5 | - | - | 6.2 | - | - |
| | 31S | 36.5 | 22.0 | - | - | - | - | - |
| | 31T | 158 | 13.6 | - | - | - | - | - |
| | 31Y | 101.7 | 13.1 | - | - | - | - | - |
| 32 | 32D | 100 | 100 | 100 | 100 | 100 | 100 | 100 |
| | 32E | 873 | 8.9 | - | - | - | - | - |
| | 32H | 0.8 | 0.1 | - | - | - | - | - |
| | 32N | 10.9 | 4.2 | 3.6 | 27.0 | 526.9 | 8.7 | 17.5 |
| | 32Q | 328.3 | 0.3 | 18.4 | - | 21.9 | - | 80.4 |
| | 32S | 167.3 | 2.9 | - | - | - | - | - |
| | 32T | 78.1 | 0.8 | - | - | - | - | - |
| | 32Y | 15.1 | 1.1 | - | - | - | - | - |
| 34 | 34A | 100 | 100 | 100 | 100 | 100 | 100 | 100 |
| | 34N | 100.1 | 7.4 | 29.2 | -9.4 | 0.7 | 29.2 | 14.4 |
| 50 | 50A | 442 | 3.8 | 95.2 | 33 | 4.3 | 17.5 | 33.4 |
| | 50D | 130.6 | 9.3 | 58.2 | | 22.2 | - | 53.0 |
| | 50E | 100 | 100 | 75.6 | 1.0 | 100 | 23.7 | 44.0 |
| | 50H | 8.8 | 4.7 | 100 | 100 | 7.3 | 100 | 100 |
| | 50Q | 51.8 | 2.9 | - | - | - | - | 61.1 |
| | 50S | 44.2 | 4.2 | - | - | - | - | 40.6 |
| | 50T | 26.9 | 3.0 | - | - | - | - | 38.8 |
| | 50Y | 46.9 | 3.8 | - | - | - | - | 74.9 |
| 53 | 53H | 80.8 | 100.2 | 115.1 | 69.6 | 94.4 | 59.5 | 75.7 |
| | 53N | 100 | 100 | 105.1 | 52.9 | 91.2 | 70.8 | 97.6 |
| | 53S | 73.6 | 101.5 | 100 | 100 | 100 | 99.0 | 73.2 |
| | 53T | 75.8 | 94.3 | 98.9 | 109.3 | 44.5 | 100 | 100 |
| 91 | 91D | 20.4 | 1.0 | - | - | - | - | - |
| | 91E | 301 | 1.1 | - | - | - | - | - |
| | 91H | 815 | 4.9 | 3201 | 21.8 | 4.5 | 77.7 | 100 |
| | 91Q | 964 | 2.9 | - | - | - | - | - |
| | 91R | 2.4 | -0.6 | -2.6 | -4.0 | 2.6 | 0.8 | 1.5 |
| | 91S | 27.3 | 1.7 | 100 | 100 | 0.6 | 100 | 17.8 |
| | 91T | 50.7 | 7.9 | - | - | - | - | - |
| | 91Y | 100 | 100 | 56.0 | 0.3 | 100 | 43.3 | 79.5 |
| 92 | 92D | 100 | 100 | 100 | 100 | 31.7 | 100 | 100 |
| | 92E | 99.8 | - | - | - | 40.9 | - | - |
| | 92N | - | 88.4 | - | 36.4 | 100 | 7.1 | 4.7 |
| | 92P | 21.7 | - | - | - | 64.5 | - | - |
| | 92Q | 59.7 | - | - | - | 124.8 | - | - |
| | 92S | 51.3 | 93.7 | 1.1 | 79.0 | 104.0 | 7.2 | 4.8 |
| | 92T | 74.1 | - | - | - | 95.1 | - | - |
| 93 | 93D | 45.1 | 76.7 | 5.4 | 22.9 | 23.8 | 66.5 | 22.2 |
| | 93G | 78.6 | 96.8 | 3.6 | 51.0 | 79.1 | 100 | 37.9 |
| | 93N | 80.4 | 96.7 | 100 | 63.0 | 107.2 | 51.9 | 28.5 |
| | 93R | 96.0 | 84.9 | 11.7 | 84.9 | 135.5 | 12.2 | 5.2 |
| | 93S | 100 | 100 | 4.0 | 100 | 100 | 33.4 | 100 |
| 94 | 94F | 106 | - | - | - | 28.6 | - | 0.9 |
| | 94H | - | - | - | - | 17.6 | - | 2.6 |
| | 94S | 100 | 102.6 | 100 | -8.4 | - | 20.9 | 0.3 |
| | 94T | 53.1 | 100.9 | 206.6 | 4.3 | - | 8.8 | 1.3 |
| | 94W | 4.5 | - | - | - | 33.7 | - | 1.3 |
| | 94Y | 46.1 | 100 | -0.7 | 100 | 100 | 100 | 100 |
| 96 | 96F | 11.8 | 67.1 | 82.8 | - | 31.8 | - | 39.9 |
| | 96H | 50.7 | 32.3 | 5.1 | - | 114.5 | - | -0.7 |
| | 96I | 102.7 | 4.5 | 0.8 | - | 56.0 | - | 19.7 |
| | 96L | 100 | 2.5 | 3.2 | 100 | 100 | 100 | 100 |
| | 96P | 49.0 | 12.5 | -1.6 | - | 18.0 | - | 4.3 |
| | 96W | 3.6 | 109.9 | 5.4 | - | 16.9 | - | 0.8 |
| | 96Y | 7.5 | 100 | 100 | -11.1 | 23.0 | 7.4 | 1.4 |
| | 96M | - | 9.1 | - | - | 46.2 | - | - |
| | 96V | - | 5.2 | - | - | 70.3 | - | - |

### Example 6

In vivo study of some anti-C5 bispecific antibodies in co-injection model

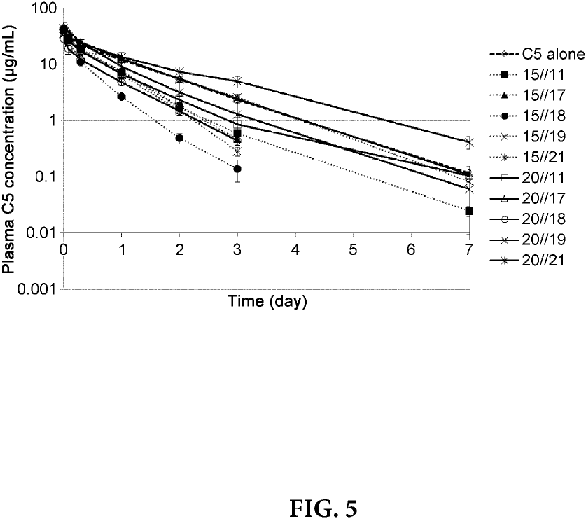

Some anti-C5 bispecific antibodies (15//11, 15//17, 15//18, 15//19, 15//21, 20//11, 20//17, 20//18, 20//19 and 20//21) comprising two distinct human engineered IgG1 constant regions from each other heavy chains (F1684mnP17 (SEQ ID NO: 49), and F1684mnN17 (SEQ ID NO: 50)) were prepared as previously described. Ten anti-C5 bispecific antibodies were tested in mice co-injection model to evaluate their ability to accelerate the clearance of C5 from plasma. In co-injection model, human FcRn transgenic mice (hFcRn-Tgm, B6.mFcRn-/-.hFcRn Tg line 276+/+ mouse, Jackson Laboratories) were administered by single i.v. injection with C5 alone or with C5 pre-mixed with anti-C5 bispecific antibody. The first group received 1.34 mg/kg C5 but the other groups additionally received 1.0 mg/kg of anti-C5 bispecific antibodies. Total C5 plasma concentration was determined by anti-C5 ECLIA. First, anti-human C5 mouse IgG was dispensed into an ECL plate, and left for overnight at 4° C. to prepare an anti-human C5 mouse IgG-immobilized plate. Samples for standard curve and samples were mixed with an anti-human C5 rabbit IgG. These samples were added into the anti-human C5 mouse IgG-immobilized plate, and left for one hour at room temperature. Then, these samples were reacted with HRP conjugated anti-rabbit IgG (Jackson Immuno Research). After the plate was incubated for one hour at room temperature, a sulfo-tag conjugated anti-HRP were added. ECL signal was read with Sector Imager 2400 (Meso Scale discovery). The concentration of human C5 was calculated from the ECL signal in the standard curve using SOFTmax PRO (Molecular Devices). FIG. 5 describes plasma concentration time profile of total C5 in human FcRn transgenic mice.

While administration of conventional antibody without pH-dependent antigen binding is known to reduce the clearance of the antigen from plasma in comparison to administration of antigen alone because antigen-antibody complex has lower clearance than the antigen itself (PLoS One. 2013 May 7;8(5):e63236), most of bispecific antibodies tested in this study demonstrated rapid C5 clearance from plasma. Among the tested antibodies, clone 20//18 were selected for further optimization.

### Example 7

### Binding Characterization and Optimization of Anti-C5 Bispecific Antibodies

### 7.1. Binding Characterization of Anti-C5 Bispecific Antibodies

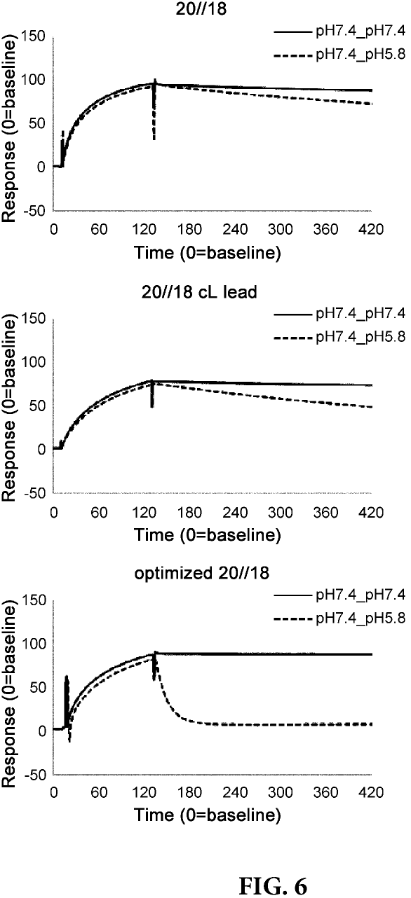

The kinetics parameters of anti-C5 bispecific antibodies, 20//18 with two different light chains and 20//18 cL lead with common light chain (amino acid sequence of these antibodies are described in Table 4), against recombinant human C5 were assessed under two different conditions (e.g. a) association and dissociation at pH 7.4 and b) association at pH 7.4 and dissociation at pH 5.8), at 37° C. using Biacore T200 instrument (GE Healthcare). Protein A/G (Pierce, Cat No. #21186) or anti-human IgG (Fc) antibody (within Human Antibody Capture Kit; GE Healthcare, Cat No. BR-1008-39) was immobilized onto a Series S CM4 (GE Healthcare, Cat No. BR-1005-34) by amine coupling method. Anti-C5 antibodies were captured on an immobilized molecule, and then human C5 was injected. The running buffers used were ACES pH 7.4 and pH 5.8 (20 mM ACES, 150 mM NaCl, 1.2 mM CaCl₂, 0.05% Tween 20). Kinetics parameters at both pH conditions were determined by fitting the sensorgrams with 1: 1 binding -RI (without bulk effect adjustment) model using Biacore T200 Evaluation software, version 2.0 (GE Healthcare). The sensorgrams of these antibodies are shown in FIG. 6. Kinetic parameters, association rate (ka), dissociation rate (kd), and binding affinity (KD) at pH 7.4, and dissociation rate (kd) determined by only calculating the dissociation phase at each pH conditions, are described in Table 5. 20//18 cL lead showed relatively slower association and dissociation rate at pH 7.4 than 20//18.

TABLE 4

Amino acid sequence of variable regions of 20//18 variants

| | | | | | | | | |
|---|---|---|---|---|---|---|---|---|
| Clone name | VH Name | VH SEQ ID | VL Name | VL SEQ ID | VH Name | VH SEQ ID | VL Name | VL SEQ ID |
| 20//18 | 20H | NO: 29 | 20L | NO: 30 | 18H | NO: 25 | 18L | NO: 26 |
| 20//18 cL lead | 20H | NO: 29 | 20L065 | NO: 35 | 18H | NO: 25 | 20L065 | NO: 35 |
| optimized 20//18 | 20H261 | NO: 52 | 20L233 | NO: 53 | 18H012 | NO: 54 | 20L233 | NO: 53 |

TABLE 5

Kinetic parameters of 20//18 variants against human C5 under two different conditions

| | | pH 7.4 | | pH 7.4 | pH 5.8 |
|---|---|---|---|---|---|
| | ka | kd | KD | kd (only dissociation) | |
| 20//18 | 9.24E+05 | 2.66E-04 | 2.88E-10 | 2.68E-04 | 8.84E-04 |
| 20//18 cL lead | 5.09E+05 | 1.86E-04 | 3.64E-10 | 2.21 E-04 | 1.48E-03 |
| optimized 20//18 | 3.62E+05 | 9.72E-05 | 2.68E-10 | 1.04E-04 | 5.94E-02 |

### 7.2. Optimization of Anti-C5 Bispecific Antibody

20//18 cL lead was further optimized to have improved binding affinity to C5 at pH 7.4 and improved pH dependency (showing more rapid dissociation at pH 5.8). Variants with amino acid substitutions introduced to both of VH and VL region were prepared by a method generally known to those skilled in the art. These variants were examined for the binding against human C5. Effective substitutions were combined to identify optimized 20//18 (amino acid sequence is described in Table 4). The optimized 20//18 was examined the binding against human C5 in the same way as described in Example 7.1 and the sensorgrams and kinetic parameters of optimized 20//18 are shown in FIG. 6 and Table 5.

### Example 8

### In Vivo Study of Fc Variants of Optimized 20//18 Bispecific Antibody in Cynomolgus Monkey

Fc variants of optimized 20//18 bispecific antibody, optimized 20//18-hIgGl (optimized clone 20-hIgG1 (20H261-G1dP1, SEQ ID NO: 55), optimized clone 18-hIgG1 (18H012-G1dN1, SEQ ID NO: 56) and optimized common Lch (20L233-k0, SEQ ID NO: 57)), -FS156 (optimized clone 20-FS156 (20H261-FS156P1, SEQ ID NO: 58), optimized clone 18-FS156 (18H012-FS156N1, SEQ ID NO: 59) and optimized common Lch (20L233-k0, SEQ ID NO: 57)) and -FS154 (optimized clone 20-FS154 (20H261-FS154P1, SEQ ID NO: 60), optimized clone 18-FS154 (18H012-FS154N1, SEQ ID NO: 61) and optimized common Lch (20L233-k0, SEQ ID NO: 57)) were prepared as previously described.

To observe the cross-reactivity of optimized 20//18 against cynomolgus monkey C5, Biacore kinetics analysis was performed in the same way as described in Example 7.1. The obtained kinetic parameters are shown in Table 6.

TABLE 6

Kinetic parameters of optimized 20//18 against cynomolgus monkey C5 under two different conditions

| | pH 7.4 | | | pH 7.4 | pH 5.8 |
|---|---|---|---|---|---|
| | ka | kd | KD | kd (only dissociation) | |
| optimized 20//18 | 3.55E+05 | 2.36E-04 | 6.64E-10 | 2.24E-04 | 1.07E-01 |

Binding affinities of hIgG1, FS156 and FS154 to cynomolgus monkey Fc gamma receptors (Fc gamma Rs) are described in Table 7. FS156 has comparable or less than 2-fold enhanced binding affinity to Fc gamma R2a and Fc gamma R2b, while significantly decreased binding affinity to Fc gamma R¹ and Fc gamma R3. FS154 has 5-10 fold enhanced binding affinity to Fc gamma R2a and Fc gamma R2b, while significantly decreased binding affinity to Fc gamma R¹ and Fc gamma R3.

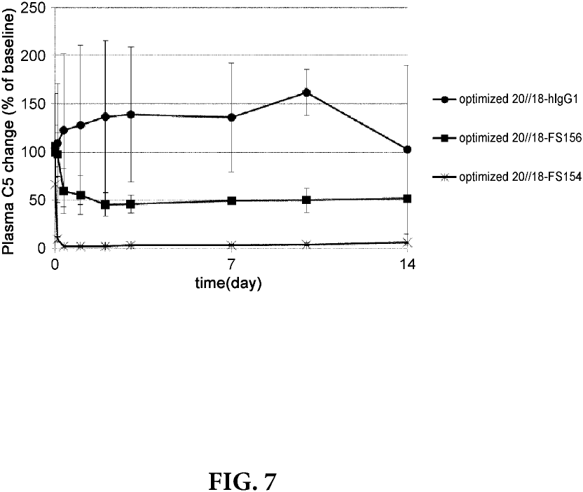

Cynomolgus monkeys were administered by single i.v. injection of anti-C5 bispecific antibodies at a dose of 10 mg/kg. Total cynomolgus monkey C5 plasma concentration was determined by anti-C5 ECLIA. First, anti-cynomolgus monkey C5 rabbit IgG was dispensed into a 96-well plate, and left for overnight at 4° C. to prepare an anti-cynomolgus monkey C5 rabbit IgG-immobilized plate. Samples for standard curve and samples were mixed with an excess anti- cynomolgus monkey C5 human IgG. These samples were added into the anti- cynomolgus monkey C5 rabbit IgG-immobilized plate, and left for one hour at room temperature. Then, these samples were reacted with a sulfo tag conjugated anti-human IgG. After the plate was incubated for one hour at room temperature, ECL signal was read with Sector Imager 2400 (Meso Scale discovery). The concentration of cynomolgus monkey C5 was calculated from the ECL signal in the standard curve using SOFTmax PRO (Molecular Devices). FIG. 7 describes plasma concentration time profile of total C5 in cynomolgus monkey.

TABLE 7

Binding affinities (KD) of hIgG1, FS156 and FS154 to cynomolgus monkey Fc gamma receptors

| | | | | | | |
|---|---|---|---|---|---|---|
| Antibody | cyFcγR1 | cyFcyR2al | cyFcγR2a2 | cyFcyR2a3 | cyFcyR2b | cyFcγR3S |
| optimized 20//18-hIgG1 | 3.67E-11 | 2.64E-06 | 1.89E-06 | 1.17E-05 | 1.32E-06 | 2.38E-07 |
| optimized 20//18-FS156 | 1.37E-09 | 3.11E-06 | 1.96E-06 | 5.96E-06 | 8.53E-07 | 3.13E-06 |
| optimized 20//18-FS154 | 2.50E-10 | 5.28E-07 | 3.41E-07 | 1.27E-06 | 2.22E-07 | 1.05E-06 |

Optimized 20//18-FS156 actively eliminated C5 from the plasma and reduced the plasma C5 concentration approximately 2-fold below the baseline; optimized 20//18-FS154 reduced plasma C5 concentration approximately 30-fold below the baseline, demonstrating that anti-C5 bispecific antibody, optimized 20//18, significantly enhanced C5 clearance in Fc gamma R2a and Fc gamma R2b dependent manner. This demonstrates that pH and/or calcium-dependent anti-C5 bispecific antibody which can form multimeric Ag-Ab IC with enhanced Fc gamma R binding is very effective approach to target C5, whose plasma concentration is very high (up to 100 micro g/mL) and requires high antibody dosage using conventional monoclonal antibody.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A combination of two or more isolated or purified anti-C5 antibodies, wherein the isolated or purified anti-C5 antibodies bind to an epitope within the beta chain (SEQ ID NO: 1) or alpha chain (SEQ ID NO: 10) of C5 and wherein the isolated or purified anti-C5 antibodies to be combined do not compete with each other for binding to the epitope.

2. The combination according to claim 1, wherein the epitope is selected from an epitope within the MG1 (SEQ ID NO: 2), MG2 (SEQ ID NO: 3), MG3 (SEQ ID NO: 4), MG4 (SEQ ID NO: 5), MG5 (SEQ ID NO: 6), MG6 (SEQ ID NO: 7), MG1-MG2 (SEQ ID NO: 8) or the MG3-MG6 (SEQ ID NO: 9) domain of the beta chain of C5, or an epitope within the anaphylatoxin domain (SEQ ID NO: 11) or C5-C345C/NTR domain (SEQ ID NO: 12) of the alpha chain of C5.

3. The combination according to claim 1 or 2, wherein the epitope is selected from within a fragment consisting of amino acids 33-124 of the beta chain (SEQ ID NO: 1) or a fragment consisting of amino acids 1-999 of the alpha chain (SEQ ID NO: 10) of C5.

4. The combination according to any one of claims 1 to 3, wherein one or more of the anti-C5 antibodies bind to C5 with a higher affinity at neutral pH than at acidic pH.

5. The combination according to any one of claims 1 to 4, wherein one or more of the isolated or purified anti-C5 antibodies bind to the same epitope as any one of reference antibodies described in Table 2.

6. The combination according to any one of claims 1 to 5, wherein one or more of the isolated or purified anti-C5 antibodies compete with any one of reference antibodies described in Table 2 for binding to C5.

7. The combination according to any one of claims 1 to 5, wherein one or more of the isolated or purified anti-C5 antibodies comprise 6 HVRs of any one of antibodies described in Table 2.

8. The combination according to any one of claims 1 to 7, wherein one or more of the isolated or purified anti-C5 antibodies modulate, inhibit, block or neutralize a biological function of C5.

9. The combination according to any one of claims 1 to 8, wherein one or more of the isolated or purified anti-C5 antibodies are a monoclonal antibody.

10. The combination according to any one of claims 1 to 9, wherein one or more of the isolated or purified anti-C5 antibodies are a human, humanized, or chimeric antibody.

11. The combination according to any one of claims 1 to 10, wherein one or more of the isolated or purified anti-C5 antibodies are a full length IgG1 or IgG4 antibody.

12. The combination according to any one of claims 1 to 11, wherein the combination of isolated or purified anti-C5 antibodies is an isolated or purified multispecific antibody.

13. A pharmaceutical formulation comprising the combination of any one of claims 1 to 12 and a pharmaceutically acceptable carrier.

14. The combination of any one of claims 1 to 11 for use as a medicament.

15. The combination of any one of claims 1 to 11 for use in treating a complement-mediated disease or condition which involves excessive or uncontrolled activation of C5.

16. The combination of any one of claims 1 to 11 for use in enhancing the clearance of C5 from plasma.

17. Use of the combination of any one of claims 1 to 11 in the manufacture of a medicament for treatment of a complement-mediated disease or condition which involves excessive or uncontrolled activation of C5.

18. Use of the combination of any one of claims 1 to 11 in the manufacture of a medicament for enhancing the clearance of C5 from plasma.

19. A method of treating an individual having a complement-mediated disease or condition which involves excessive or uncontrolled activation of C5, the method comprising administering to the individual an effective amount of the combination of any one of claims 1 to 11.

20. A method of enhancing the clearance of C5 from plasma in an individual comprising administering to the individual an effective amount of the combination of any one of claims 1 to 11 to enhance the clearance of C5 from plasma.
